(19) United States
(12) Patent Application Publication
(10) Pub. No.: US 2007/0196856 A1
(43) Pub. Date: Aug. 23, 2007

(54) Methods of determining activity of ryanodine receptor modulators

(75) Inventors: Cun-Jian Dong, Irvine, CA (US); William A. Hare, Tustin, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 11/362,319

(22) Filed: Feb. 23, 2006

(51) Int. Cl. G01N 33/53; A61K 31/727; A61K 31/522; A61K 31/4745; A61K 33/40; G06T 7/00

(52) U.S. Cl. 435/7.1; 514/56; 514/263.34; 514/26; 424/616; 514/291; 382/120

(57) ABSTRACT

Methods for identifying modulators of ryanodine receptors are disclosed. In preferred embodiments the activity of the ryanodine receptor is stimulated to a baseline level and the ability of a test compound to increase or decrease the baseline level indicates that the test compound is a modulator of ryanodine receptor activity.

### BACKGROUND AND SUMMARY OF THE INVENTION

The present invention relates to methods of identifying ryanodine receptor modulators. More particularly, the invention includes test procedures that may be used to identify novel compounds that can increase, block, or decrease the activity of ryanodine receptors.

Abnormal release of Ca⁺⁺ (calcium ion) from ryanodine receptors (RyRs) is believed to contribute to intracellular Ca⁺⁺ overload and stress to the endoplasmic reticulum (ER) that can lead to neuronal cell injury in a number of neurological disorders, such as glaucoma, amyotrophic lateral sclerosis, Alzheimer's disease, and Parkinson's disease, as well as stroke and acute brain trauma. Thus, it would be advantageous to provide substances which are effective to modulate, for example, increase or decrease, release of Ca⁺⁺ from ryanodine receptors.

To this end, new methods for screening substances for effectiveness as ryanodine receptor modulators would be beneficial.

Methods for determining the ability of a test substance to modulate the activity of a ryanodine receptor have been discovered. The present methods allow for high throughput screening of potential ryanodine receptor modulators, for example, using conventional imaging techniques and multi-well test plates. The present methods are relatively easy to practice, and provide reliable information and results useful in identifying one or more test substances having beneficial ryanodine receptor activity modulation properties.

Ryanodine receptors are members of a superfamily of Ca⁺⁺ release channels that also include the inositol 1,4,5-triphosphate receptors. In particular, the ryanodine receptors are calcium induced, calcium release channels that play a critical role in most cells, including muscle cells, neurons and epithelial cells. They mediate the release of calcium ion from the endoplasmic (sarcoplasmic) reticulum (“ER” or “SR”, respectively) into the cytoplasm; thus the ryanodine receptors are commonly found in the membrane of the ER.

RyR has been purified, cloned, and sequenced from a variety of species, and several isoforms have been identified. Mammalian tissues express three isoforms, known as RyR1, RyR2, and RyR3. They include about 5000 (4872 to 5037) amino acid residues and are encoded by three different genes. In humans, the three genes are located on chromosomes 19, 1, and 15, respectively. RyR1 and RyR2 are expressed predominantly in skeletal muscle and in cardiac muscle, respectively (Marks et al., PROC. NATL. ACAD. SCI. USA 86: 8683-8687, 1989; Takeshima et al., NATURE (Lond.) 339: 439-445, 1989; Nakai et al., FEBS LETT. 271: 169-177, 1990; Otsu et al., J. BIOL. CHEM. 265: 13472-13483, 1990; Zorzato et al., J. BIOL. CHEM. 265: 2244-2256, 1990). RyR3 has a wide tissue distribution, although it has been originally identified in brain and is sometimes called “brain isoform.” All three isoforms are actually expressed in brain, and the major brain isoform does not appear to be RyR3, but rather RyR2. Alternative splicing variants of RyR1 and RyR2 have been identified, but their functional relevance remains to be established (Sutko and Airey, PHYSIOL. REV. 76: 1027-1071, 1996). Two RyR isoforms, known as α-RyR and β-RyR, have been identified in fish, amphibian, and avian skeletal muscle, and they are the homologues of mammalian RyR1 and RyR3, respectively). The overall identity of the RyR isoforms is of the order of 66 to 67%.

The RyR selectively binds the plant alkaloid ryanodine, which is the reason for its name. In keeping with the RyRs other name, the endoplasmic reticulum calcium channel, Ca⁺⁺ is thought to be the “physiological” channel activator, because other ligands either cannot activate the channel in the absence of Ca⁺⁺ or they require Ca⁺⁺ for maximum effect.

Existing methods of studying RyR modulation include, (see e.g., Zuchhi et al., PHARM. REV. 49:1 (1997)) include the following:

1) isolating sarcoplasmic reticulum (SR) vesicles containing the RyR and loading them with Ca⁺⁺. Ca⁺⁺ release is then induced using a release solution (such as one containing ryanodine) and measuring the extravesicular Ca⁺⁺ flux following induction.

2) Using SR vesicles or purified RyRs incorporated into artificial lipid bilayers. When these bilayers separate two ionic solutions, current flow between the two chambers indicates the presence of the calcium channel. Prospective modulators can be added to the “extracellular” chamber and current recordings can monitor changes in the conductivity.

3) Labeled ryanodine binding to the RyR. The affinity of ryanodine to the receptor can be affected by the functional state of the RyR.

4) Indirect studies using tension development (contractile response) in isolated or skinned muscle cells after exposure to caffeine or a prospective ligand can be interpreted as an index of Ca⁺⁺ release. However, this is not always the case as these ligands can have other targets beyond the RyR receptor. Additionally, other sarcoplasmic or intracellular transporters can affect C++ release.

Other methods for studying RyR biochemistry have employed cloned receptors. Thus, in Bhat et al., BIOPHYS. J. 77:808 (August 1999) rabbit cardiac muscle RyR (RyR1 and RyR2) was cloned and transfected into Chinese hamster ovary (CHO) cells and Ca⁺⁺ release from these cells upon exposure to caffeine was studied. Similarly, in Xiao et al., J. BIOL. CHEM. 277:41778 (2002), human embryonic kidney cells (HEK293) cells were transfected with the three RyR isoforms, RyR1, RyR2 and RyR3, as well as mutant RyR receptors.

RyR1 has been observed to form homotetramers when isolated from rabbit skeletal muscle. In the Xiao study cited above the authors found, using immunoprecipitation and co-expression studies, that RyR2 was able to interact with RyR1 and RyR3 in HEK cells thereby forming heterotetramers, but that RyR1 does not interact with RyR3, even when co-expressed in the same cell or tissue. Thus, RyR1 and RyR3 appear to exist only in a homotetrameric form in the absence of RyR2.

The present invention is based upon the finding that modulators of one or more functional RyR calcium channel can be assayed in a cell by stimulating a baseline level of calcium release using a known ryanodine receptor activating component such as caffeine, then adding a potential RyR modulator with the known agonist to determine its effect on caffeine-inducted Ca⁺⁺ release through RyR. In this way antagonists, inverse agonists and agonists of the selected RyR channel can be identified.

By “ryanodine receptor activating component” in the present specification is meant a compound or substance known to bind to and stimulate the Ca⁺⁺ releasing activity of the ryanodine receptor.

By “test substance” is meant a compound or substance whose activity, or extent of activity, at one or more ryanodine receptor subtype is sought to be determined, verified, or compared with other test substances, with a ryanodine receptor activating component, or with a ryanodine receptor inhibiting component.

By “ryanodine receptor inhibiting component” is meant a compound that either block activation of a RyR receptor isoform in the presence of a ryanodine receptor activating component, or which decreases a baseline level of activity of a RyR receptor isoform in the absence of a ryanodine receptor activating component or another ryanodine receptor inhibiting component.

Using cloned RyR receptor isoforms, modulators of desired RyR channels (such as homotetrameric channels comprising only one of RyR1, RyR2 or RyR3) can be identified; alternatively any mixture of RyR isoforms (such as RyR2+RyR1 or RyR2+RyR3) can be co-expressed and the effect of prospective modulators of heteromeric calcium channels can be studied. In a preferred embodiment, Ca⁺⁺ flux can be detected and measured using, for example, a membrane permeable Ca⁺⁺ selective fluorescent dye such as fluo-4 AM. In this system, the cell cultures can be illuminated at a wavelength of about 488 nm and fluorescence monitored and measured at a wavelength of about 520 nm. A variety of fluorescent dyes suitable for measuring Ca⁺⁺ flux are available from various suppliers including the Molecular Probes division of Invitrogen, Inc.; these may include, without limitation, fura-2, indo-1, quin-2, quin-2 AM, fura-4F, fura-5F and fura-6F, fura-FF, fluo-3, rhod-2, rhod-FF, calcium green-1, calcium green-2, calcium yellow, calcium orange, calcium crimson, Oregon-green, BAPTA-1, BAPTA-6F, and conjugates, such as dextran linked conjugates of one or more such dyes. Different dyes or probes may have different absorption and/or emission maxima; some are designed to be detected within the visible light spectrum, others are designed to be detected at wavelengths outside that of visible light, such as in the UV range.

### BRIEF DESCRIPTION OF THE DRAWINGS

FIG. 1 is a plot demonstrating an embodiment of the assay of the present invention. An increase in fluorescent intensity (monitoring of the fluo-45 dye at or near its emission maximum) indicates an increase in cytosolic free Ca⁺⁺ concentration. Under control conditions, extracellular application of 1.5 mM caffeine elicited a significant increase of cytosolic free Ca⁺⁺ (the trace identified by the numeral 1). This caffeine-induced Ca⁺⁺ release was blocked by 20 mM dantrolene (see the trace identified by 2). The caffeine effect was recovered partially after washout with 12.5 mM caffeine alone (the trace marked 3). The upward deflection 4 of the horizontal line 5 above the response traces indicates the duration of caffeine application.

Thus, in one broad aspect of the present invention, methods for determining the ability of a test substance to modulate the activity of a ryanodine receptor are provided. Such methods comprise contacting a ryanodine receptor in a cell with an effective amount of a ryanodine receptor activating component and a test substance; and monitoring the release of Ca⁺⁺ in the cell. In one embodiment, the methods further comprise comparing the release of Ca⁺⁺ in the cell with a control release of Ca⁺⁺ in a substantially identical cell substantially identically contacted without the test substance. By comparing the Ca⁺⁺ release with and without the test substance one can reliably determine the ability, for example, qualitatively and/or quantitatively, of the test substance to modulate the activity of the ryanodine receptor.

In another broad aspect of the present invention, methods for determining the ability of a test substance to modulate the activity of a ryanodine receptor are provided and comprise the following steps A, B and C. In step A, a first ryanodine receptor in a first cell is contacted with a first activating component in a dose effective to stimulate Ca⁺⁺ release by the ryanodine receptor and the release of Ca⁺⁺ is monitored. In step B, a second ryanodine receptor in a second cell is contacted with a second activating component in a substantially equivalent dose to the dose of the first activating component used in step A and a test substance. The release, if any, of Ca⁺⁺ by the second ryanodine receptor is monitored. The first and second ryanodine receptors are substantially identical and the first and second cells are from substantially the same cell line. Additionally, the first and second activating components are substantially identical. In step C, the releases of Ca⁺⁺ in step A and in step B are compared.

The difference in the releases of Ca⁺⁺ in step A and in step B is an indication of the ability of the test substance to modulate ryanodine receptor activity. Thus, such method provides a useful tool in determining the ability, for example, qualitatively and/or quantitatively of the test substance to modulate ryanodine receptor activity. Moreover, in preferred embodiments the assay is capable of being carried out quickly in a high throughput format and is amenable to automation of one or more, preferably substantially all steps.

The first cell and the second cell, for example, the cell and the substantially identical cell, are advantageously from the same cell line, and may preferably be clones.

In one embodiment, the contacting steps and monitoring steps are carried out a statistically significant number of times, either in terms of numbers of identical samples, or in terms of repetitive assays using the same cells and/or test substances. Thus, the contacting and monitoring steps using identical concentrations of a given test substance may be performed in duplicate, triplicate, quadruplicate, and the like. Additionally, assays of the same test substance may be conducted at different concentrations in order to obtain a statistically significant dose-response curve. The present methods are very useful when applied to high throughput screening assays. In particular, the present contacting and monitoring steps advantageously are carried out automatically, for example robotically.

The monitoring step may be carried out in any suitable manner. In one useful embodiment, the monitoring step comprises monitoring calcium release by way of an electromagnetic signal, for example, a light based signal monitored within a given wavelength range. Common wavelength ranges are within the visible or UV spectra. Additionally, the light based signal may, for example, vary within a given dynamic range in response to the extent of the Ca⁺⁺ release by the ryanodine receptor. The signal may be a fluorescence signal, although other types of electromagnetic signals may be employed. When fluorescence dyes are used, generally the cell will be illuminated with light at one wavelength at or near the absorption maximum for the dye, and monitored for fluorescent emission at a different wavelength at or near the emission maximum for such dye.

During the contacting step, the cell may, and advantageously does, include a Ca⁺⁺ indicator. For example, the Ca⁺⁺ indicator may be permeable to the membrane of the cell and be contained within the cell.

The Ca⁺⁺ indicator may be a component effective to have a detectably altered state in the presence of Ca⁺⁺ relative to a base state in the absence of Ca⁺⁺. The Ca⁺⁺ indicator may comprise a fluorescence indicator, for example, comprising fluo-4-AM, the like indicators and mixtures thereof.

The test substance may be any substance for which it is desired to determine the ability to modulate the activity of a ryanodine receptor. Such test substance may be selected from ryanodine receptor agonists, ryanodine receptor antagonists, ryanodine receptor inverse agonists and the like, or from any substance whose potential activity as a ryanodine receptor agonist, ryanodine receptor antagonist, ryanodine receptor inverse agonist or ryanodine receptor co-modulator is sought to be determined. In one embodiment, the test substance binds to at least one ryanodine receptor isoform selected from the group consisting of RyR1, RyR2 and RyR3. In a further embodiment the test substance binds to at least two, or at least three of these receptor isoforms.

Any substance which is effective to activate Ca⁺⁺ release in a ryanodine receptor may be used as the activating component. In one useful embodiment the ryanodine receptor-activating component comprises a caffeine component. Such caffeine component may be selected, for example, from caffeine, caffeine analogs, caffeine derivatives and mixtures thereof. Other known ryanodine receptor activating components comprise, without limitation, inorganic phosphate; adenine nucleotides; adenosine; cADPR; paslitoyl carnitate; protein kinase A; calmodulin; ryanodine; methylxanthines other than caffeine and caffeine analogs and derivatives; anthriquinones; digoxin; milrinone; suramin; halothine; enflurine; isoflurine; 4-chloro-m-cresol, δ-hexachlorocyclohexane; FK-506; rapamycin; bastadin 5; quinolidomicin A1; heparin; imperitoxin-a; miotoxin a; ryanotoxin; thimerisol; dithiodipyridine; hydrogen peroxide; TMPyP; disulfonic stilbene derivatives; and diethylpyrocarbonate.

The monitoring step may comprise detecting Ca⁺⁺ release using a charge coupled device (CCD) camera (CCD technology is adapted for producing high-resolution images in conditions of ultra low light), a photomultiplier tube (PMT) and the like. The monitoring may comprise Ca⁺⁺ imaging, for example, fluorescent Ca⁺⁺ imaging. In one useful embodiment, the contacting and monitoring steps are conducted using contacting and monitoring steps in both the substantial absence and presence of the test substance.

The RyR receptor isoforms used in the assays of the present invention are preferably human in origin, although RyR isoforms from, for example, rabbit, porcine, and bullfrog origin have very similar amino acid sequences as compared to human counterparts of a given RyR receptor and may be used as a substitute therefor. Additionally, this fact seems to suggest that the amino acid sequences of the RyRs are quite highly conserved between species generally.

Preferably the assay employs RyR1, RyR2 or RyR3, which have respective GenBank accession numbers P21817 (and NP_{—}000531), 092736 and (NP_{—}001026), and 015413 (and (NP_{—}001027). Rabbit and porcine RyR1 have GenBank accession numbers P11716 and P16960, respectively. Rabbit RyR2 has GenBank accession number P30957 and Ry44 (analogous to human RyR3) has GenBank accession number 024498. The accession numbers for all of these sequences, and a Blast alignment showing similarities between selected sequences, were obtained on Dec. 21, 2005.

These sequences are as follows:

Ryanodine receptor 1 (homo sapiens)
Accession No. NP 000531 (SEQ ID NO: 1)
MGDAEGEDEV QFLRTDDEVV LQCSATVLKE QLKLCLAAEG
FGNRLCFLEP TSNAQNVPPD LAICCFVLEQ SLSVRALQEM
LANTVEAGVE SSQGGGHRTL LYGHAILLRH AHSRMYLSCL
TTSRSMTDKL AFDVGLQEDA TGEACWWTMH PASKQRSEGE
KVRVGDDIIL VSVSSERYLH LSTASGELQV DASFMQTLWN
MNPICSRCEE GFVTGGHVLR LEHGHMDECL TISPADSDDQ
RRLVYYEGGA VCTHARSLWR LEPLRISWSG SHLRWGQPLR
VRHVTTGQYL ALTEDQGLVV VDASKAHTKA TSFCFRISKE
KLDVAPKRDV EGMGPPEIKY GESLCFVQHV ASGLWLTYAA
PDPKALRLGV LKKKAMLHQE GHMDDALSLT RCQQEESQAA
RMIHSTNGLY NQFIKSLDSF SGKPRGSGPP AGTALPIEGV
ILSLQDLIIY FEPPSEDLQH EEKQSKLRSL RNRQSLFQEE
GMLSMVLNCI DRLNVYTTAA HFAEFAGEEA AESWKEIVNL
LYELLASLIR GNRSNCALFS TNLDWLVSKL DRLEASSGIL
EVLYCVLIES PEVLNIIQEN HIKSIISLLD KHGRNHKVLD
VLCSLCVCNG VAVRSNQDLI TENLLPGREL LLQTNLINYV
TSIRPNIFVG RAEGTTQYSK WYFEVMVDEV TPFLTAQATH
LRVGWALTEG YTPYPGAGEG WGGNGVGDDL YSYGFDGLHL
WTGHVARPVT SPGQHLLAPE DVISCCLDLS VPSISFRING
CPVQGVFESF NLDGLFFPVV SFSAGVKVRF LLGGRHGEFK
FLPPPGYAPC HEAVLPRERL HLEPIKEYRR EGPRGPHLVG
PSRCLSHTDF VPCPVDTVQI VLPPHLERIR EKLAENIHEL
WALTRIEQGW TYGPVRDDNK RLHPCLVDFH SLPEPERNYN
LQMSGETLKT LLALGCHVGM ADEKAEDNLK KTKLPKTYMM
SNGYKPAPLD LSHVRLTPAQ TTLVDRLAEN GHNVWARDRV
GQGWSYSAVQ DIPARRNPRL VPYRLLDEAT KRSNRDSLCQ
AVRTLLGYGY NIEPPDQEPS QVENQSRCDR VRIFRAEKSY
TVQSGRWYFE FEAVTTGEMR VGWARPELRP DVELGADELA
YVFNGHRGQR WHLGSEPFGR PWQPGDVVGC MIDLTENTII
FTLNGEVLMS DSGSETAFRE IEIGDGFLPV CSLGPGQVGH
LNLGQDVSSL RFFAICGLQE GFEPFAINMQ RPVTTWFSKG
LPQFEPVPLE HPHYEVSRVD GTVDTPPCLR LTHRTWGSQN
SLVEMLFLRL SLPVQFHQHF RCTAGATPLA PPGLQPPAED
EARAAEPDPD YENLRRSAGG WSEAENGKEG TAKEGAPGGT
PQAGGEAQPA RAENEKDATT EKNKKRGFLF KAKKVAMMTQ
PPATPTLPRL PHDVVPADNR DDPEIILNTT TYYYSVRVFA
GQEPSCVWAG WVTPDYHQHD MSFDLSKVRV VTVTMGDEQG
NVHSSLKCSN CYMVWGGDFV SPGQQGRISH TDLVIGCLVD
LATGLMTFTA NGKESNTFFQ VEPNTKLFPA VFVLPTHQNV
IQFELGKQKN IMPLSAAMFQ SERKNPAPQC PPRLEMQMLM
PVSWSRMPNH ELQVETRRAG ERLGWAVQCQ EPLTMMALHI
PEENRCMDIL ELSERLDLQR FHSHTLRLYR AVCALGNNRV
AHALCSHVDQ AQLLHALEDA HLPGPLRAGY YDLLISIHLE
SACRSRRSML SEYIVPLTPE TRAITLFPPG RSTENGHPRH
GLPGVGVTTS LRPPHHFSPP CFVAALPAAG AAEAPARLSP
AIPLEALRDK ALRMLGEAVR DGGQHARDPV GASVEFQFVP
VLKLVSTLLV MGIFGDEDVK QILKMIEPEV FTEEEEEEDE
EEEGEEEDEE EKEEDEEETA QEKEDEEKEE EEAAEGEKEE
GLEEGLLQMK LPESVKLQMC HLLEYFCDQE LQHRVESLAA
FAERYVDKLQ ANQRSRYGLL IKAFSMTAAE TARRTREFRS
PPQEQINMLL QFKDGTDEED CPLPEEIRQD LLDFHQDLLA
HCGIQLDGEE EEPEEETTLG SRLMSLLEKV RLVKKKEEKP
EEERSAEESK PRSLQELVSH MVVRWAQEDF VQSPELVRAM
FSLLHRQYDG LGELLRALPR AYTISPSSVE DTMSLLECLG
QIRSLLIVQM GPQEENLMIQ SIGNIMNNKV FYQHPNLMRA
LGMHETVMEV MVNVLGGGES KEIRFPKMVT SCCRFLCYFC
RISRQNQRSM FDHLSYLLEN SGIGLGMQGS TPLDVAAASV
IDNNELALAL QEQDLEKVVS YLAGCGLQSC PMLVAKGYPD
IGWNPCGGER YLDFLRFAVF VNGESVEENA NVVVRLLIRK
PECFGPALRG EGGSGLLAAI EEAIRISEDP ARDGPGIRRD
RRREHFGEEP PEENRVHLGH AIMSFYAALI DLLGRCAPEM
HLIQAGKGEA LRIRAILRSL VPLEDLVGII SLPLQIPTLG
KDGALVQPKM SASEVPDHKA SMVLFLDRVY GIENQDFLLH
VLDVGFLPDM RAAASLDTAT FSTTEMALAV NRYLCLAVLP
LITKCAPLFA GTEHRAIMVD SMLHTVYRLS RGRSLTKAQR
DVIEDCLMSL CRYIRPSMLQ HLLRRLVFDV PILNEFAKMP
LKLLTNHYER CWKYYCLPTG WANFGVTSEE ELHLTRKLFW
GIFDSLAHKK YDPELYRMAM PCLCAIAGAL PPDYVDASYS
SKAEKKATVD AEGNFDPRPV ETLNVIIPEK LDSFINKFAE
YTHEKWAFDK IQNNWSYGEN IDEELKTHPM LRPYKTFSEK
DKEIYRWPIK ESLKAMIAWE WTIEKAREGE EEKTEKKKTR
KISQSAQTYD PREGYNPQPP DLSAVTLSRE LQAMAEQLAE
NYHNTWGRKK KQELEAKGGG THPLLVPYDT LTAKEKARDR
EKAQELLKFL QMNGYAVTRG LKDMELDSSS IEKRFAFGFL
QQLLRWMDIS QEFIAHLEAV VSSGRVEKSP HEQEIKFFAK
ILLPLINQYF TNHCLYFLST PAKVLGSGGH ASNKEKEMIT
SLFCKLAALV RHRVSLFGTD APAVVNCLHI LARSLDARTV
MKSGPEIVKA GLRSFFESAS EDIEKMVENL RLGKVSQART
QVKGVGQNLT YTTVALLPVL TTLFQHIAQH QFGDDVILDD
VQVSCYRTLC SIYSLGTTKN TYVEKLRPAL GECLARLAAA
MPVAFLEPQL NEYNACSVYT TKSPRERAIL GLPNSVEEMC
PDIPVLERLM ADIGGLAESG ARYTEMPHVI EITLPMLCSY
LPRWWERGPE APPSALPAGA PPPCTAVTSD HLNSLLGNIL
RIIVNNLGID EASWMKRLAV FAQPIVSRAR PELLQSHFIP
TIGRLRKRAG KVVSEEEQLR LEAKAEAQEG ELLVRDEFSV
LCRDLYALYP LLIRYVDNNR AQWLTEPNPS AEELFRMVGE
IFIYWSKSHN FKREEQNFVV QNEINNMSFL TADNKSKMAK
AGDIQSGGSD QERTKKKRRG DRYSVQTSLI VATLKKMLPI
GLNMCAPTDQ DLITLAKTRY ALKDTDEEVR EFLHNNLHLQ
GKVEGSPSLR WQMALYRGVP GREEDADDPE KIVRRVQEVS
AVLYYLDQTE HPYKSKKAVW HKLLSKQRRR AVVACFRMTP
LYNLPTHRAC NMFLESYKAA WILTEDHSFE DRMIDDLSKA
GEQEEEEEEV EEKKPDPLHQ LVLHFSRTAL TEKSKLDEDY
LYMAYADIMA KSCHLEEGGE NGEAEEEVEV SFEEKQMEKQ
RLLYQQARLH TRGAAEMVLQ MISACKGETG AMVSSTLKLG
ISILNGGNAE VQQKMLDYLK DKKEVGFFQS TQALMQTCSV
LDLNAFERQN KAEGLGMVNE DGTVINRQNG EKVMADDEFT
QDLFRFLQLL CEGHNNDFQN YLRTQTGNTT TINIIICTVD
YLLRLQESIS DFYWYYSGKD VIEEQGKRNF SKAMSVAKQV
FNSLTEYIQG PCTGNQQSLA HSRLWDAVVG FLHVFAHMMM
KLAQDSSQIE LLKELLDLQK DMVVMLLSLL EGNVVNGMIA
RQMVDMLVES SSNVEMILKF FDMFLKLKDI VGSEAFQDYV
TDPRGLISKK DFQKAMDSQK QFSGPEIQFL LSCSEADENE
MINCEEFANR FQEPARDIGF NVAVLLTNLS EHVPHDPRLH
NFLELAESIL EYFRPYLGRI EIMGASRRIE RIYFEISETN
RAQWEMPQVK ESKRQFIFDV VNEGGEAEKM ELFVSFCEDT
IFEMQIAAQI SEPEGEPETD EDEGAGAAEA GAEGAEEGAA
GLEGTAATAA AGATARVVAA AGRALRGLSY RSLRRRVRRL
RRLTAREAAT AVAALLWAAV TRAGAAGAGA AAGALGLLWG
SLFGGGLVEG AKKVTVTELL AGMPDPTSDE VHGEQPAGPG
GDADGEGASE GAGDAAEGAG DEEEAVHEAG PGGADGAVAV
TDGGPFRPEG AGGLGDMGDT TPAEPPTPEG SPILKRKLGV
DGVEEELPPE PEPEPEPELE PEKADAENGE KEEVPEPTPE
PPKKQAPPSP PPKKEEAGGE FWGELEVQRV KFLNYLSRNF
YTLRELALFL AFAINFILLF YKVSDSPPGE DDMEGSAAGD
VSGAGSGGSS GWGLGAGEEA EGDEDENMVY YPLEESTGYM
EPALRCLSLL HTLVAFLCII GYNCLKVPLV IFKREKELAR
KLEFDGLYIT EQPEDDDVKG QWDRLVLNTP SFPSNYWDKF
VKRKVLDKHG DIYGRERIAE LLGMDLATLE ITAHNERKPN
PPPGLLTWLM SIDVKYQIWK FGVIFTDNSF LYLGWYMVMS
LLGHYNNFFF AAHLLDIAMG VKTLRTILSS VTHNGKQLVM
TVGLLAVVVY LYTVVAFNFF RKFYNKSEDE DEPDMKCDDM
MTCYLFHMYV GVRAGGGIGD EIEDPAGDEY ELYRVVFDIT
FFFFVIVILL AIIQGLIIDA EGELRDQQEQ VKEDMETKCF
ICGIGSDYFD TTPHGFETHT LEEHNLANYM FFLMYLINKD
ETEHTGQESY VWKMYQERCW DFFPAGDCFR KQYEDQLS

Ryanodine Receptor 2 (Homo Sapiens) GenBank
Accession No. NP_001026 has the following
amino acid sequence (SEQ ID NO: 2):
MADGGEGEDE IQFLRTDDEV VLQCTATIHK EQQKLCLAAE
GFGNRLCFLE STSNSKNVPP DLSICTFVLE QSLSVRALQE
MLANTVEKSE GQVDVEKWKF MMKTAQGGGH RTLLYGHAIL
LRHSYSGMYL CCLSTSRSST DKLAFDVGLQ EDTTGEACWW
TIHPASKQRS EGEKVRVGDD LILVSVSSER YLHLSYGNGS
LHVDAAFQQT LWSVAPISSG SEAAQGYLIG GDVLRLLHGH
MDECLTVPSG EHGEEQRRTV HYEGGAVSVH ARSLWRLETL
RVAWSGSHIR WGQPFRLRHV TTGKYLSLME DKNLLLMDKE
KADVKSTAFT FRSSKEKLDV GVRKEVDGMG TSEIKYGDSV
CYIQHVDTGL WLTYQSVDVK SVRMGSIQRK AIMHHEGHMD
DGTSLSRSQH EESRTARVIR STVFLFNRFI RGLDALSKKA
KASTVDLPIE SVSLSLQDLI GYFHPPDEHL EHEDKQNRLR
ALKNRQNLFQ ESGMINLVLE CIDRLHVYSS AAHFADVAGR
EAGESWKSIL NSLYELLAAL IRGNRKNCAQ FSGSLDWLIS
RLERLEASSG ILEVLHCVLV ESPEALNIIK EGHIKSIISL
LDKHGRNHKV LDVLCSLCVC HGVAVRSNQH LICDNLLPGR
DLLLQTRLVN HVSSMRPNIF LGVSEGSAQY KKWYYELMVD
HTEPFVTAEA THLRVGWAST EGYSPYPGGG EEWGGNGVGD
DLFSYGFDGL HLWSGCIART VSSPNQHLLR TDDVISCCLD
LSAPSISFRI NGQPVQGMFE NFNIDGLFFP VVSESAGIKV
RFLLGGRHGE FKPLPPPGYA PCYEAVLPKE KLKVEHSREY
KQERTYTRDL LGPTVSLTQA AFTPIPVDTS QIVLPPHLER
IREKLAENIH ELWVMNKIEL GWQYGPVRDD NKRQHPCLVE
FSKLPEQERN YNLQMSLETL KTLLALGCHV GISDEHAEDK
VKKMKLPKNY QLTSGYKPAP MDLSFIKLTP SQEAMVDKLA
ENAHNVWARD RIRQGWTYGI QQDVKNRRNP RLVPYTPLDD
RTKKSNKDSL REAVRTLLGY GYNLEAPDQD HAARAEVCSG
TGERFRIFRA EKTYAVKAGR WYFEFETVTA GDMRVGWSRP
GCQPDQELGS DERAFAFDGF KAQRWHQGNE HYGRSWQAGD
VVGCMVDMNE HTMMFTLNGE ILLDDSGSEL AFKDFDVGDG
FIPVCSLGVA QVGRMNFGKD VSTLKYFTIC GLQEGYEPFA
VNTNRDITMW LSKRLPQFLQ VPSNHEHIEV TRIDGTIDSS
PCLKVTQKSF GSQNSNTDIM FYRLSMPIEC AEVFSKTVAG
GLPGAGLFGP KNDLEDYDAD SDFEVLMKTA HGHLVPDRVD
KDKEATKPEF NNHKDYAQEK PSRLKQRFLL RRTKPDYSTS
HSARLTEDVL ADDRDDYDFL MQTSTYYYSV RIFPGQEPAN
VWVGWITSDF HQYDTGFDLD RVRTVTVTLG DEKGKVHESI
KRSNCYMVCA GESMSPGQGR NNNGLEIGCV VDAASGLLTF
IANGKELSTY YQVEPSTKLF PAVFAQATSP NVFQFELGRI
KNVMPLSAGL FKSEHKNPVP QCPPRLHVQF LSHVLWSRMP
NQELKVDVSR ISERQGWLVQ CLDPLQFMSL HIPEENRSVD
ILELTEQEEL LKFHYHTLRL YSAVCALGNH RVAHALCSHV
DEPQLLYAIE NKYMPGLLRA GYYDLLIDIH LSSYATARLM
MNNEYIVPMT EETKSITLFP DENKKHGLPG IGLSTSLRPR
MQFSSPSFVS ISNECYQYSP EFPLDILKSK TIQMLTEAVK
EGSLHARDPV GGTTEFLFVP LTKLFYTLLI MGIFHNEDLK
HILQLIEPSV FKEAATPEEE SDTLEKELSV DDAKLQGAGE
EEAKGGKRPK EGLLQMKLPE PVKLQMCLLL QYLCDCQVRH
RIEAIVAFSD DFVAKLQDNQ RFRYNEVMQA LNMSAALTAR
KTKEFRSPPQ EQINMLLNFK DDKSECPCPE EIRDQLLDFH
EDLMTHCGIE LDEDGSLDGN SDLTIRGRLL SLVEKVTYLK
KKQAEKPVES DSKKSSTLQQ LISETMVRWA QESVIEDPEL
VRAMFVLLHR QYDGIGGLVR ALPKTYTING VSVEDTINLL
ASLGQIRSLL SVRMGKEEEK LMIRGLGDIM NNKVFYQHPN
LMRALGMHET VMEVMVNVLG GGESKEITFP KMVANCCRFL
CYFCRISRQN QKAMFDHLSY LLENSSVGLA SPAMRGSTPL
DVAAASVMDN NELALALREP DLEKVVRYLA GCGLQSCQML
VSKGYPDIGW NPVEGERYLD FLRFAVFCNG ESVEENANVV
VRLLIRRPEC FGPALRGEGG NGLLAAMEEA IKIAEDPSRD
GPSPNSGSSK TLDTEEEEDD TIHMGNAIMT FYSALIDLLG
RCAPEMHLIH AGKGEAIRIR SILRSLIPLG DLVGVISIAF
QMPTIAKDGN VVEPDMSAGF CPDHKAAMVL FLDRVYGIEV
QDFLLHLLEV GFLPDLRAAA SLDTAALSAT DMALALNRYL
CTAVLPLLTR CAPLFAGTEH HASLIDSLLH TVYRLSKGCS
LTKAQRDSIE VCLLSICGQL RPSMMQHLLR RLVEDVPLLN
EHAKMPLKLL TNHYERCWKY YCLPGGWGNF GAASEEELHL
SRKLEWGIFD ALSQKKYEQE LFKLALPCLS AVAGALPPDY
MESNYVSMME KQSSMDSEGN FNPQPVDTSN ITIPEKLEYF
INKYAEHSHD KWSMDKLANG WIYGEIYSDS SKVQPLMKPY
KLLSEKEKEI YRWPIKESLK TMLARTMRTE RTREGDSMAL
YNRTRRTSQT SQVSVDAAHG YSPRAIDMSN VTLSRDLHAM
AEMMAENYHN IWAKKKKMEL ESKGGGNHPL LVPYDTLTAK
EKAKDREKAQ DILKFLQING YAVSRGFKDL ELDTPSIEKR
FAYSFLQQLI RYVDEAHQYI LEFDGGSRGK GEHFPYEQEI
KFFAKVVLPL IDQYFKNHRL YFLSAASRPL CSGGHASNKE
KEMVTSLFCK LGVLVRHRIS LFGNDATSIV NCLHILGQTL
DARTVMKTGL ESVKSALRAF LDNAAEDLEK TMENLKQGQF
THTRNQPKGV TQTINYTTVA LLPMLSSLFE HIGQHQFGED
LILEDVQVSC YRILTSLYAL GTSKSIYVER QRSALGECLA
AFAGAFPVAF LETHLDKHNI YSIYNTKSSR ERAALSLPTN
VEDVCPNIPS LEKLMEEIVE LAESGIRYTQ MPHVMEVILP
MLCSYMSRWW EHGPENNPER AEMCCTALNS EHMNTLLGNI
LKIIYNNLGI DEGAWMKRLA VFSQPIINKV KPQLLKTHFL
PLMEKLKKKA ATVVSEEDHL KAEARGDMSE AELLILDEFT
TLARDLYAFY PLLIRFVDYN RAKWLKEPNP EAEELFRMVA
EVEIYWSKSH NFKREEQNFV VQNEINNMSF LITDTKSKMS
KAAVSDQERK KMKRKGDRYS MQTSLIVAAL KRLLPIGLNI
CAPGDQELIA LAKNRFSLKD TEDEVRDIIR SNIHLQGKLE
DPAIRWQMAL YKDLPNRTDD TSDPEKTVER VLDIANVLFH
LEQKSKRVGR RHYCLVEHPQ RSKKAVWHKL LSKQRKRAVV
ACFRMAPLYN LPRHRAVNLF LQGYEKSWIE TEEHYFEDKL
IEDLAKPGAE PPEEDEGTKR VDPLHQLILL FSRTALTEKC
KLEEDFLYMA YADIMAKSCH DEEDDDGEEE VKSFEEKEME
KQKLLYQQAR LHDRGAAEMV LQTISASKGE TGPMVAATLK
LGIAILNGGN STVQQKMLDY LKEKKDVGFF QSLAGLMQSC
SVLDLNAFER QNKAEGLGMV TEEGSGEKVL QDDEFTCDLF
RFLQLLCEGH NSDFQNYLRT QTGNNTTVNI IISTVDYLLR
VQESISDFYW YYSGKDVIDE QGQRNFSKAI QVAKQVFNTL
TEYIQGPCTG NQQSLAHSRL WDAVVGFLHV FAHMQMKLSQ
DSSQIELLKE LMDLQKDMVV MLLSMLEGNV VNGTIGKQMV
DMLVESSNNV EMILKFFDMF LKLKDLTSSD TFKEYDPDGK
GVISKRDFHK AMESHKHYTQ SETEFLLSCA ETDENETLDY
EEFVKRFHEP AKDIGFNVAV LLTNLSEHMP NDTRLQTFLE
LAESVLNYFQ PFLGRIEIMG SAKRIERVYF EISESSRTQW
EKPQVKESKR QFIFDVVNEG GEKEKMELFV NFCEDTIFEM
QLAAQISESD LNERSANKEE SEKERPEEQG PRMAFFSILT
VRSALFALRY NILTLMRMLS LKSLKKQMKK VKKMTVKDMV
TAFFSSYWSI FMTLLHFVAS VERGFTRIIC SLLLGGSLVE
GAKKIKVAEL LANMPDPTQD EVRGDGEEGE RKPLEAALPS
EDLTDLKELT EESDLLSDIF GLDLKREGGQ YKLIPHNPNA
GLSDLMSNPV PMPEVQEKFQ EQKAKEEEKE EKEETKSEPE
KAEGEDGEKE EKAKEDKGKQ KLRQLHTHRY GEPEVPESAF
WKKIIAYQQK LLNYFARNFY NMRMLALFVA FAINFILLFY
KVSTSSVVEG KELPTRSSSE NAKVTSLDSS SHRIIAVHYV
LEESSGYMEP TLRILAILHT VISFFCIIGY YCLKVPLVIF
KREKEVARKL EFDGLYITEQ PSEDDIKGQW DRLVINTQSF
PNNYWDKFVK RKVMDKYGEF YGRDRISELL GMDKAALDFS
DAREKKKPKK DSSLSAVLNS IDVKYQMWKL GVVFTDNSFL
YLAWYMTMSV LGHYNNFFFA AHLLDIAMGF KTLRTILSSV
THNGKQLVLT VGLLAVVVYL YTVVAFNFFR KFYNKSEDGD
TPDMKCDDML TCYMFHMYVG VRAGGGIGDE IEDPAGDEYE
IYRIIFDITF FFFVIVILLA ITQGLIIDAF GELRDQQEQV
KEDMETKCFI CGIGNDYFDT VPHGFETHTL QEHNLANYLF
FLMYLINKDE TEHTGQESYV WKMYQERCWE FFPAGDCERK
QYEDQLN

Human Ryanodine Receptor 3 (Homo Sapiens)
GenBank Accession No. NP_001027 has the
following sequence (SEQ ID NO: 3):
MAEGGEGGED EIQFLRTEDE VVLQCIATIH KEQRKFCLAA
EGLGNRLCFL EPTSEAKYIP PDLCVCNFVL EQSLSVRALQ
EMLANTGENG GEGAAQGGGH RTLLYGHAVL LRHSESGMYL
TCLTTSRSQT DKLAFDVGLR EHATGEACWW TIHPASKQRS
EGEKVRIGDD LILVSVSSER YLHLSVSNGN IQVDASFMQT
LWNVHPTCSG SSIEEGYLLG GHVVRLFHGH DECLTIPSTD
QNDSQHRRIF YEAGGAGTRA RSLWRVEPLR ISWSGSNIRW
GQAFRLRHLT TGHYLALTED QGLILQDRAK SDTKSTAFSF
RASKELKEKL DSSHKRDIEG MGVPEIKYGD SVCFVQHIAS
GLWVTYKAQD AKTSRLGPLK RKVILHQEGH MDDGLTLQRC
QREESQAARI IRNTTALFSQ FVSGNNRTAA PITLPIEEVL
QTLQDLIAYF QPPEEEMRHE DKQNKLRSLK NRQNLFKEEG
MLALVLNCID RLNVYNSVAH FAGIAREESG MAWKEILNLL
YKLLAALIRG NRNNCAQFSN NLDWLISKLD RLESSSGILE
VLHCILTESP EALNLIAEGH IKSIISLLDK HGRNHKVLDI
LCSLCLCNGV AVRANQNLIC DNLLPRRNLL LQTRLINDVT
SIRPNIFLGV AEGSAQYKKW YFELIIDQVD PFLTAEPTHL
RVGWASSSGY APYPGGGEGW GGNGVGDDLY SYGFDGLHLW
SGRIPRAVAS TNQHLLRSDD VVSCCLDLGV PSTSFRINGQ
PVQGMFENFN TDGLFFPVMS FSAGVKVRFL MGGRHGEFKF
LPPSGYAPCY EALLPKEKMR LEPVKEYKRD ADGIRDLLGT
TQFLSQASFI PCPVDTSQVI LPPHLEKIRD RLAENIHELW
GMNKIELGWT FGKIRDDNKR QHPCLVEFSK LPETEKNYNL
QMSTETLKTL LALGCHIAHV NPAAEEDLKK VKLPKNYMMS
NGYKPAPLDL SDVKLLPPQE ILVDKLAENA HNVWAKDRIK
QGWTYGIQQD LKNKRNPRLV PYALLDERTK KSNRDSLREA
VRTFVGYGYN IEPSDQELAD SAVEKVSIDK IRFFRVERSY
AVRSGKWYFE FEVVTGGDMR VGWARPGCRP DVELGADDQA
FVFEGNRGQR WHQGSGYFGR TWQPGDVVGC MINLDDASMI
FTLNGELLIT NKGSELAFAD YEIENGFVPI CCLGLSQIGR
MNLGTDASTF KFYTMCGLQE GFEPFAVNMN RDVAMWFSKR
LPTFVNVPKD HPHIEVMRID GTMDSPPCLK VTHKTFGTQN
SNADMIYCRL SMPVECHSSF SHSPCLDSEA FQKRKQMQEI
LSHTTTQCYY AIRIFAGQDP SCVWVGWVTP DYHLYSEKFD
LNKNCTVTVT LGDERGRVHE SVKRSNCYMV WGGDIVASSQ
RSNRSNVDLE IGCLVDLAMG MLSFSANGKE LGTCYQVEPN
TKVFPAVFLQ PTSTSLFQFE LGKLKNAMPL SAAIFRSEEK
NPVPQCPPRL DVQTIQPVLW SRMPNSFLKV ETERVSERHG
WVVQCLEPLQ MMALHIPEEN RCVDILELCE QEDLMRFHYH
TLRLYSAVCA LGNSRVAYAL CSHVDLSQLF YAIDNKYLPG
LLRSGPYDLL ISIHLASAKE RKLMMKNEYI IPITSTTRNI
CLFPDESKRH GLPGVGLRTC LKPGFRFSTP CFVVTGEDHQ
KQSPEIPLES LRTKALSMLT EAVQCSGAHI RDPVGGSVEF
QFVPVLKLIG TLLVMGVFDD DDVRQILLLI DPSVFGEHSA
GTEEGAEKEE VTQVEEKAVE AGEKAGKEAP VKGLLQTRLP
ESVKLQMCEL LSYLCDCELQ HRVEAIVAFG DIYVSKLQAN
QKFRYNELMQ ALNMSAALTA RKTKEFRSPP QEQINMLLNF
QLGENCPCPE EIREELYDFH EDLLLHCGVP LEEEEEEEED
TSWTGKLCAL VYKIKGPPKP EKEQPTEEEE RCPTTLKELI
SQTMICWAQE DQIQDSELVR MMFNLLRRQY DSIGELLQAL
RKTYTISHTS VSDTTNLLAA LGQIRSLLSV RMGKEEELLM
INGLGDIMNN KVFYQHPNLM RVLGMHETVM EVMVNVLGTE
KSQIAFPKMV ASCCRELCYF CRISRQNQKA MFEHLSYLLE
NSSVGLASPS MRGSTPLDVA ASSVMDNNEL ALSLEEPDLE
KVVTYLAGCG LQSCPMLLAK GYPDVGWNPI EGERYLSFLR
FAVFVNSESV EENASVVVKL LIRRPECFGP ALRGEGGNGL
LAAMQGAIKI SENPALDLPS QGYKREVSTE DDEEEEEIVH
MGNAIMSFYS ALTDLLGRCA PEMHLIQTGK GEAIRIRSIL
RSLVPTEDLV GIISIPLKLP SLNKDGSVSE PDMAANFCPD
HKAPMVLFLD RVYGIKDQTF LLHLLEVGFL PDLRASASLD
TVSLSTTEAA LALNRYICSA VLPLLTRCAP LFAGTEHCTS
LIDSTLQTIY RLSKGRSLTK AQRDTIEECL LAICNHLRPS
MLQQLLRRLV FDVPQLNEYC KMPLKLLTNH YEQCWKYYCL
PSGWGSYGLA VEEELHLTEK LFWGIFDSLS HKKYDPDLFR
MALPCLSAIA GALPPDYLDT RITATLEKQI SVDADGNFDP
KPTNTMNFSL PEKLEYIVTK YAEHSHDKWA CDKSQSGWKY
GISLDENVKT HPLIRPFKTL TEKEKEIYRW PARESLKTML
AVGWTVERTK EGEALVQQRE NEKLRSVSQA NQGNSYSPAP
LDLSNVVLSR ELQGMVEVVA ENYHNIWAKK KKLELESKGG
GSHPLLVPYD TLTAKEKFKD REKAQDLFKF LQVNGIIVSR
GMKDMELDAS SMEKRFAYKF LKKILKYVDS AQEFIAHLEA
IVSSGKTEKS PRDQEIKFFA KVLLPLVDQY FTSHCLYFLS
SPLKPLSSSG YASHKEKEMV AGLFCKLAAL VRHRTSLFGS
DSTTMVSCLH ILAQTLDTRT VMKSGSELVK AGLRAFFENA
AEDLEKTSEN LKLGKETHSR TQIKGVSQNI NYTTVALLPI
LTSIFEHVTQ HQFGMDLLLG DVQISCYHIL CSLYSLGTGK
NIYVERQRPA LGECLASLAA AIPVAFLEPT LNRYNPLSVF
NTKTPRERSI LGMPDTVEDM CPDIPQLEGL MKEINDLAES
GARYTEMPHV IEVILPMLCN YLSYWWERGP ENLPPSTGPC
CTKVTSEHLS LILGNILKII NNNLGIDEAS WMKRTAVYAQ
PIISKARPDL LRSHFTPTLE KLKKKAVKTV QEEEQLKADG
KGDTQEAELL ILDEFAVLCR DLYAFYPMLI RYVDNNRSNW
LKSPDADSDQ LERMVAEVFI LWCKSHNFKR EEQNFVIQNE
TNNLAFLTGD SKSKMSKAMQ VKSGGQDQER KKTKRRGDLY
SIQTSLIVAA LKKMLPIGLN MCTPGDQELI SLAKSRYSHR
DTDEEVREHL RNNLHLQEKS DDPAVKWQLN LYKDVLKSEE
PFNPEKTVER VQRISAAVFH LEQVEQPLRS KKAVWHKLLS
KQRKRAVVAC FRMAPLYNLP RHRSINLFLH GYQRFWIETE
EYSFEEKLVQ DLAKSPKVEE EEEEETEKQP DPLHQIILYF
SRNALTERSK LEDDPLYTSY SSMMAKSCQS GEDEEEDEDK
EKTFEEKEME KQKTLYQQAR LHERGAAEMV LQMISASKGE
MSPMVVETLK LGIAILNGGN AGVQQKMLDY LKEKKDAGFF
QSLSGLMQSC SVLDLNAFER QNKAEGLGMV TEEGTLIVRE
RGEKVLQNDE FTRDLFRFLQ LLCEGHNSDF QNFLRTQMGN
TTTVNVIIST VDYLLRLQES ISDFYWYYSG KDIIDESGQH
NFSKALAVTK QIFNSLTEYI QGPCIGNQQS LAHSRLWDAV
VGFLHVFANM QMKLSQDSSQ IELLKELLDL LQDMVVMLLS
LLEGNVVNGT IGKQMVDTLV ESSTNVEMIL KFFDMFLKLK
DLTSSDTFKE YDPDGKGIIS KKEFQKAMEG QKQYTQSEID
FLLSCAEADE NDMFNYVDFV DRFHEPAKDI GFNVAVLLTN
LSEHMPNDSR LKCLLDPAES VLNYFEPYLG RIEIMGGAKK
IERVYFEISE SSRTQWEKPQ VKESKRQFIF DVVNEGGEQE
KMELFVNFCE DTIFEMQLAS QISESDSADR PEEREEDEDS
SYVLEIAGEE EEDGSLEPAS AFAMACASVK RNVTDFLKRA
TLKNLRKQYR NVKKMTAKEL VKVLFSFFWM LFVGLFQLLF
TILGGIFQIL WSTVFGGGLV EGAKNIRVTK ILGDMPDPTQ
FGIHDDTMEA ERAEVMEPGI TTELVHFIKG EKGDTDIMSD
LFGLHPKKEG SLKHGPEVGL GDLSEIIGKD EPPTLESTVQ
KKRKAQAAEM KAANEAEGKV ESEKADMEDG EKEDKDKEEE
QAEYLWTEVT KKKKRRCGQK VEKPEAFTAN FPKGLEIYQT
KLLHYLARNF YNLRFLALFV AFAINFTLLF YKVTEEPLEE
ETEDVANLWN SFNDEEEEEA MVFFVLQEST GYMAPTLRAL
AIIHTIISLV CVVGYYCLKV PLVVFKREKE IARKLEFDGL
YITEQPSEDD IKGQWDRLVI NTPSFPNNYW DKFVKRKVIN
KYGDLYGAER IAELLGLDKN ALDFSPVEET KAEAASLVSW
LSSIDMKYHI WKLGVVFTDN SFLYLAWYTT MSVLGHYNNF
FFAAHLLDIA MGFKTLRTIL SSVTHNGKQL VLTVGLLAVV
VYLYTVVAFN FFRKFYNKSE DDDEPDMKCD DMMTCYLFHM
YVGVRAGGGI GDEIEDPAGD PYEMYRIVFD ITFFFFVIVI
LLAIIQGLII DAFGELRDQQ EQVREDMETK CFICGIGNDY
FDTTPHGFET HTLQEHNLAN YLFFLMYLIN KDETEHTGQE
SYVWKMYQER CWDFFPAGDC FRKQYEDQLG

Any and all patents, publications, patent applications, and nucleotide and/or amino acid sequences referred to by accession numbers cited in this specification are hereby incorporated by reference as part of this specification.

Each and every feature described herein, and each and every combination of two or more of such features, is included within the scope of the present invention provided that the features included in such a combination are not mutually inconsistent.

These and other aspects of the present invention are set forth in the following detained description, examples, and claims. The following non-limiting examples illustrate certain aspects of the invention.

### EXAMPLE 1

Vector Construction

The ryanodine receptors RyR1, RyR2 and RyR3 may be cloned in the following manner, which is indicated for RyR1. A commercially available vector, pcDNA3, is purchased from Invitrogen Corp., San Diego, Calif. This eukaryotic/prokaryotic shuttle vector or plasmid, which is 5.4 kb in length, includes the following elements: the cytomegalovirus (CMV) eukaryotic promoter and the T7 bacteriophage promoter, both promoting transcription in the clockwise direction; the SP6 bacteriophage promoter, promoting transcription in the opposite direction; a polylinker containing restriction sites for, in order from 5′ to 3′ with respect to the cloned sequences described below,: Hind III, Kpn I, Bam H1, BstX I, EcoR I, EcoR V, BstX I, Not I, XhoI, Xba I and Apa I; the SV40 eukaryotic origin of replication, the Co1E1 bacterial episomal origin of replication, the ampicillin resistance gene, and the neomycin resistance gene.

This plasmid is linearized using the restriction enzymes Not I and Bam I as follows. A 200 μl reaction mixture containing 300 μg/ml pcNDA3 DNA, 600 units/ml each of Not I and Bam I (Invitrogen, Inc.), 10 mM Tris HCl (pH 7.9), 10 mM MgCl₂, 50 mM NaCl, 1 mM dithiolthreitol (DTT) and 100 μg/ml BSA (bovine serum albumin) is incubated at 37° C. overnight. The DNA fragments are separated on a 1% agarose gel using TBE (89 mM Tris (pH 8.0), 89 mM boric acid, and 2 mM EDTA (ethylene diamine tetraacetic acid)). The large linearized DNA fragment is excised from the gel. The gel slice is crushed and the DNA is extracted by adsorption on glass particles, and purified by precipitation in ethanol. The purified DNA fragment is resuspended in TE (10 mM Tris (pH 7.5, 1 mM EDTA), and the concentration of the purified DNA fragment ascertained by determining the absorbance of the solution at 260 nm in a spectrophotometer. The isolated DNA is stored at −20° C. until use.

### EXAMPLE 2

Cloning of Ryanodine Receptor into pcDNA 3

The DNA encoding the ryanodine receptor is obtained from PCR amplification of total RNA (mRNA) cDNA from human skeletal muscle cells. For RyR2, cardiac muscle cells may be used, and brain tissue may be used for the isolation of RyR3 mRNA. RNA is collected from the muscle cells using standard and well-known procedures. The RNA is reverse transcribed in a reaction mixture containing 1 μg muscle cell whole RNA, 12.5 mM each dNTP, 50 mM Tris-HCl (pH 8.3), 40 mM KCl, 5 mM DTT (dithiolthreitol), 20 pmoles of a random deoxyribonucleotide hexamer, and 100 units SUPERSCRIPT® reverse transcriptase. The reaction mixture is incubated at 42° C. for 1 hour, then at 95° C. for 5 minutes, and stored at 4° C. until use.

PCR reactions of the cDNA preparation are performed using appropriate oligonucleotide primers complementary to (or identical to) either the 5′ or 3′ portion of the RyR1 mRNA nucleotide sequence. The sense primer incorporates a ATG start codon and a Bam HI site into the amplified nucleic acid.

The PCR reaction is set up by adding the following reagents to a sterile 0.6 ml microfuge tube in the following order: ten microliters of 10×PCR Buffer II (100 mM Tris HCl (pH 8.3), 500 mM KCl), 6 μl of 25 mM MgCl₂ 2 μl of a 10 mM solution of each dNTP, 2.5 μl of 10 μM sense primer, 2.5 μl of 10 μM antisense primer, 0.5 μl (2.5 units) of AMPLITAQ® thermostable DNA polymerase (Perkin Elmer Corp.), 66 μl ultra pure water, and one wax bead. The reaction mixture is incubated at 70° C. until the wax bead melted, then 10 μl of the skeletal muscle total RNA cDNA is added. The reaction mixture is placed in a Perkin Elmer 480 Thermal Cycler, and the cycler programmed to run 30 cycles under the following conditions: 1 minute at 94° C., 55° C. for 1 minute, 72° C. for 1.5 minutes, and at 4° C. until use.

The amplified DNA from the PCR reaction is gel purified by electrophoresis through a 1% agarose gel in TBE. The DNA band corresponding to the amplified DNA is excised from the gel, and eluted in 40 μl of water as above.

The ryanodine fragment and the linearized pcDNA vector fragment are each digested with BamHI and Not I, and the larger DNA fragments of each reaction are gel purified. The purified ryanodine receptor fragment and vector fragment are then ligated together.

The ligation reaction is performed in a total volume of 20 μg 1 containing approximately 100 ng pcDNA3 and 100 ng of the ryanodine receptor PCR fragment. This is incubated in 50 mM Tris-HCl (pH 7.8), 10 mM MgCl₂, 10 mM DTT, 1 mM ATP, 25 μg/mL BSA with 1 unit of DNA ligase at room temperature overnight.

The resulting expression vector is termed pRYAN01, having the ryanodine fragment in the proper orientation. Vector construction is confirmed by diagnostic restriction digestion and nucleic acid sequencing. Large scale vector preparations are made from the transformed E. coli clone.

### EXAMPLE 3

Transfection of Cells with pRYAN01 and Expression of the Protein

The host cells chosen to demonstrate expression of the chimeric protein of the present invention are HEK293 cells. This cell line is known to express functional RyR proteins and can be used for large scale RyR modulator screening by transfection and expression of a recombinant vector such as pRYAN01, that encodes RyR1.

HEK293 cells are grown in Dulbecco's Modified Eagle Medium supplemented with 4500 mg/nl D glucose, 584 mg/ml L-glutamine, and 10% fetal bovine serum (FBS). For transformations, cells are seeded at 1-2×10⁵ cells/ml and incubated at 37° C. at 5% CO₂ until 50-70% confluent. By percentage confluent is meant the percentage of the substrate, such as the microtiter dish bottom, that is occupied by cells.

The cells are then transfected as follows. For each transfection a solution is made by mixing 20 μl LIPOFECTIN® (a cationic lipid preparation containing a 1:1 molar ratio of DOTMA (N->1-(2-,3-dioleyloxy)propyl-N,N,N trimethylammonium chloride) and DOPE (dioleyl phosphatidylethanolamine) with 100 μl serum-free medium and the solution is allowed to stand at room temperature for 30 minutes. One to two microliters of the pRYAN01 solution is also diluted into 100 μl serum-free medium. The two solutions are combined, mixed gently and incubated at room temperature for 10-15 minutes. Cells are then overlayed with the DNA-LIPOFECTIN® mixture and incubated overnight at 37° C. The transfection mixture is then removed and replaced with medium. Expression of the pRYAN01 vector is constitutive in the HEK293 cells.

### EXAMPLE 4

Ca⁺⁺ Release from Ryanodine Receptors

The ability of a selected modulator (test substance) of the ryanodine receptor was used to model the assay of the present invention in the rat retinal ganglion cell as follows.

Rabbit retina was isolated from rabbit eyes using standard techniques, and was maintained in Ames' medium (Sigma Aldrich) during the course of the experiment. The cells were provided intracellularly with a calcium-sensitive fluorescent dye (Fluo-4®) using a patch clamp electrode. The structure of this dye, which can be purchased from the Molecular Probes division of Invitrogen, Inc., is as follows:

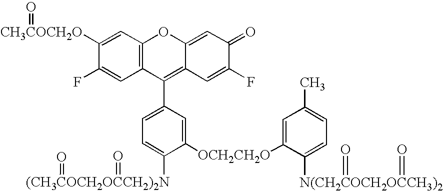

The isolated retina was placed in a recording chamber and superfused continuously with Ames' medium. Caffeine and dantrolene were was delivered briefly (i.e., approximately 10 seconds) to each cell tested through a computer-controlled multichannel rapid local perfusion system using a micro pipette which is 100-200 microns in diameter and was positioned close to the ganglion cells being recorded. In the tests where dantrolene was applied the ganglion cells were pretreated with dantrolene through the bath perfusion for 5 minutes before co-application of caffeine and dantrolene through the local perfusion started. and controlled by computer using multichannel delivery system; as were the test substances.

Images of the illuminated cells are captured with a intensified charge-coupled device (CCD) camera; intensified CCD technology is adapted for producing high-resolution images in conditions of ultra low light. Images are collected at the rate of 120 images/minute (2 images per second).

This assay seeks to determine the effect of a test substance on Ca⁺⁺ release from ryanodine receptors. Changes in intracellular free Ca⁺⁺ concentration are monitored with a fluorescent Ca⁺⁺ dye, for example, Fluo-4, in the rat ganglion cells tested.

Dantrolene (a hydantoin derivative muscle relaxant used as a treatment for malignant hyperthermia) is known to function by depressing excitation-contraction coupling in skeletal muscle by binding to the ryanodine receptor, and decreasing intracellular calcium. Dantrolene (“DTL”) is thus known to be effective in blocking caffeine-induced Ca⁺⁺ release from intracellular stores by ryanodine receptors. This compound is used as the test substance in the assay described above, which is run using 1) 1.5 mM caffeine (a ryanodine receptor activator that induces Ca⁺⁺ release from intracellular stores through the ryanodine receptor), 2) 1.5 mM+20 mM DTLM, or 3) cells given 1.5 mM caffeine+20 mM DTM, followed by a wash of the cells with 12.5 mM caffeine alone.

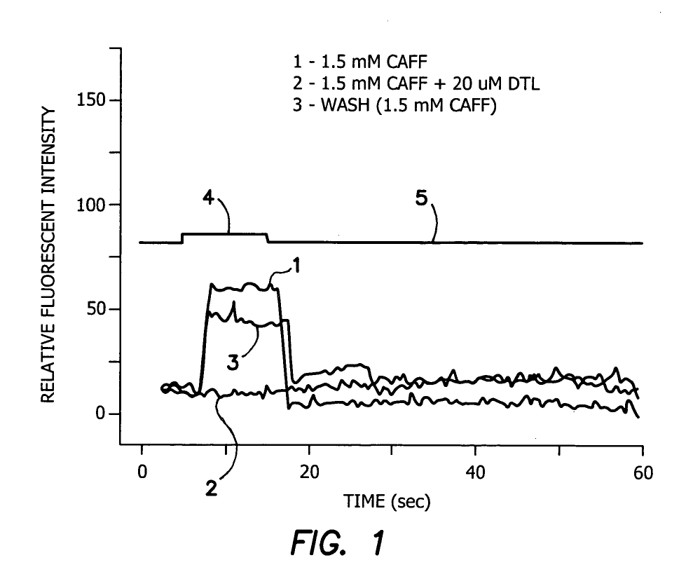

Results of this assay are discussed with reference to FIG. 1, in which the y-axis is relative fluorescent intensity (arbitrary units), and the x-axis is time.

An increase in fluorescent intensity (monitoring of the fluo-45 dye at or near its emission maximum indicates an increase in cytosolic free Ca⁺⁺ concentration. Under control conditions, extracellular application of caffeine elicited a significant increase of cytosolic free Ca⁺⁺ (the trace identified by the numeral 1). This caffeine-induced Ca⁺⁺ release was blocked by dantrolene (see the trace identified by 2). The caffeine effect was recovered partially after washout (the trace marked 3). The upward deflection 4 of the horizontal line 5 above the response traces indicates the duration of drug application.

Similar results were observed in all 5 retinal ganglion cells tested.

### EXAMPLE 5

Automation of RyR Assay

The present assay is amenable to complete or partial automation. In non-automated assays, generally speaking (and without limitation), chemists create libraries of compounds (such as, without limitation, combinatorial libraries) and biologists and medicinal chemists use them in experiments to try to understand complex biological systems. The chemical libraries are formatted in 96 or 384-well microwell plates with each well containing a small volume of compound—typically 10 to 40 μL. Researchers who desire to screen these libraries using a given assay format must develop their assays in 96 or 384 well assay plate format, and dispense their cells or protein into plates under exacting conditions. Laboratory staff is then required to transfer a small volume of a solution containing the test substance (for example, 100 nL) from the library to the assay plates. Often this transfer is accomplished using steel pin arrays. The final step in the procedure is to read out the plates in a manner consistent with the assay method, for example, using a spectrophotometric, or PMT plate reader or a CCD microscope and to interpret the results.

Automation of the present assay is carried out as follows: cultures of HEK293 cells expressing RyR1 are dispensed using a robotic manifold dispenser and accompanying software, purchased from a commercial supplier (Examples of such suppliers are CRS Ultra High Throughput Screening System, Hudson Control Group, Inc. of Springfield, N.J.). The manifold dispenser has 16 channels and is capable of filling each 384-well plate in as little as 15 seconds while pipetting accurately a volume as little as 5 μL per well.

Transfer of test substances is performed using an automated “pin transfer” step. The pins are carefully machined from stainless steel and are affixed to an adapter plate in an array that allows each pin to be centered over each well of the 384-well plate containing different test substances+1.5 mM caffeine, and control wells containing 1.5 mM caffeine only. The pins are dipped into the library plate and 100 nL is transferred into the assay plate containing 30 μL of RyR expressing HEK293 cells in culture media. Test compounds are serially diluted such that concentrations are in a range covering three orders of magnitude from 10 nM to 10 μM. The pins are washed in methanol and water between transfers.

The pin transfer and liquid handling steps are performed using a robotic platform having a large deck for setting out library and assay plates for transfer, a 4-axis robotic arm specifically designed by the manufacturer to handle microwell plates. The arm moves the library and assay plates from microplate stacks to two pin transfer positions on the deck and back. The platform has an integrated liquid handlers, a CCD camera plate reader, and a barcode reader with the system. A computer records the CCD data and correlates each dataset with a barcode identifying the corresponding well. Up to a 100,000 data points per day can be analyzed using this system.

The data received using this automated assay indicates that Ca⁺⁺ release by the RyR is stimulated by the presence of caffeine, and that the caffeine response is lowered noticeably in the presence of dandrolene and certain other test substances, while the caffeine response is augmented in the presence of other test substances. The identified modulators of the caffeine response are selected for further study.

While this invention has been described with respect to various specific examples and embodiments, it is to be understood that the invention is not limited thereto and that it can be variously practiced within the scope of the following claims.

What is claimed is:

1. A method for determining the ability of a test substance to modulate the activity of a ryanodine receptor (RyR) isoform, the method comprising:
contacting a RyR isoform in a cell with an effective amount of a ryanodine receptor activating component and a test substance; and
monitoring the release of Ca⁺⁺ by the RyR isoform.

2. The method of claim 1, which further comprises comparing the release of Ca⁺⁺ by said RyR isoform with a control release of Ca⁺⁺ in a substantially identical cell substantially identically contacted with an effective amount of a ryanodine receptor activating component in the absence of the test substance.

3. The method of claim 2, wherein the cell and the substantially identical cell are from the same cell line.

4. The method of claim 2, wherein the cell and the substantially identical cell are clones.

5. The method of claim 2, wherein the difference in the release of Ca⁺⁺ resulting from the contacting including the test substance and the control release of Ca⁺⁺ is an indication of the ability of the test substance to modulate ryanodine receptor activity.

6. The method of claim 1, wherein the contacting step and monitoring step are performed more than once for each test substance.

7. The method of claim 1, wherein the contacting step and monitoring step are performed on differing concentrations of the same test substance.

8. The method of claim 1, wherein the contacting step and monitoring step are performed more than once for the same concentration of each substance.

9. The method of claim 1, wherein at least one of the contacting step and monitoring step are automated.

10. The method of claim 1, wherein at least one of the contacting step and the monitoring step are carried out robotically.

11. The method of claim 1, wherein the monitoring comprises monitoring an electromagnetic emission signal from said cell.

12. The method of claim 11, wherein the electromagnetic emission signal varies in response to the extent of the release of Ca⁺⁺ by said ryanodine receptor isoform.

13. The method of claim 11, wherein the electromagnetic emission signal is a fluorescent signal.

14. The method of claim 1, wherein during the contacting step the cell includes a Ca⁺⁺ indicator.

15. The method of claim 14, wherein the Ca⁺⁺ indicator is permeable to the membrane of the cell.

16. The method of claim 14, wherein the Ca⁺⁺ indicator is a component effective to have a detectably altered state in the presence of Ca⁺⁺ relative to a base state in the absence of Ca⁺⁺.

17. The method of claim 16 wherein the response of said cell to changes in intracellular Ca⁺⁺ flux is measured quantitatively as a function of test substance concentration.

18. The method of claim 14, wherein the Ca⁺⁺ indicator comprises a fluorescent indicator.

19. The method of claim 14, wherein the Ca⁺⁺ indicator comprises a compound selected from the group consisting of fura-2, indo-1, fluo-4, fluo-4 AM, quin-2, quin-2 AM, fura-4F, fura-5F and fura-6F, fura-FF, fluo-3, rhod-2, rhod-FF, calcium green-1, calcium green-2, calcium yellow, calcium orange, calcium crimson, Oregon-green, BAPTA-1, BAPTA-6F, and conjugates comprising one or more such dyes.

20. The method of claim 1, wherein the test substance is selected from the group consisting of ryanodine receptor agonists, ryanodine receptor antagonists, and ryanodine receptor inverse agonists.

21. The method of claim 1, wherein the test substance binds to the ryanodine receptor isoform.

22. The method of claim 1, wherein the ryanodine receptor activating component is selected from the group consisting of caffeine; inorganic phosphate; adenine nucleotides; adenosine; cADPR; paslitoyl carnitate; protein kinase A; calmodulin; ryanodine; methylxanthines other than caffeine; anthriquinones; digoxin; milrinone; suramin; halothine; enflurine; isoflurine; 4-chloro-m-cresol, δ-hexachlorocyclohexane; FK-506; rapamycin; bastadin 5; quinolidomicin A1; heparin; imperitoxin-a; miotoxin a; ryanotoxin; thimerisol; dithiodipyridine; hydrogen peroxide; TMPyP; disulfonic stilbene; and diethylpyrocarbonate, and derivatives and analogs of these compounds.

23. The method of claim 22, wherein the ryanodine receptor activating component is selected from the group consisting of caffeine, caffeine analogs, caffeine derivatives and mixtures thereof.

24. The method of claim 1, wherein the monitoring comprises detecting Ca⁺⁺ released using a CCD camera or a PMT.

25. The method of claim 1, wherein the monitoring comprises Ca⁺⁺ imaging.

26. The method of claim 1, wherein the monitoring comprises fluorescent Ca⁺⁺ imaging.

27. The method of claim 1, wherein, after the contacting and monitoring steps, repeating the contacting and monitoring steps in the substantial absence of the test substance.

28. A method for determining the ability of a test substance to modulate the activity of a ryanodine receptor isoform, the method comprising:
(A) contacting a first ryanodine receptor isoform in a first cell with a first activating component in a dose effective to stimulate Ca⁺⁺ release by the ryanodine receptor isoform, and monitoring the release of Ca⁺⁺;
(B) contacting a second ryanodine receptor isoform in a second cell with a second activating component in a substantially equivalent dose to the dose of the first activating component used in step (A) and a test substance, and monitoring the release of Ca⁺⁺, wherein the first and second ryanodine receptors isoforms are substantially identical and the first and second cells are from substantially the same cell line; and
(C) comparing the releases of Ca⁺⁺ in step (A) and step (B).

29. The method of claim 28, wherein the difference in the releases of Ca⁺⁺ in step (A) and step (B) is an indication of the ability of the test substance to modulate ryanodine receptor activity.

30. The method of claim 28, wherein the contacting step and monitoring step are performed more than once for each test substance.

31. The method of claim 28, wherein the contacting step and monitoring step are performed on differing concentrations of the same test substance.

32. The method of claim 28, wherein the contacting step and monitoring step are performed more than once for the same concentration of each substance.

33. The method of claim 28, wherein at least one of steps (A) and (B) are automated.

34. The method of claim 28, wherein the monitoring of at least one of steps (A) and (B) comprises monitoring a electromagnetic emission signal.

35. The method of claim 34, wherein the electromagnetic signal varies in response to the amount of Ca⁺⁺ released.

36. The method of claim 34, wherein the light-based signal is a fluorescence signal.

37. The method of claim 28, wherein the monitoring of each of steps (A) and (B) comprises monitoring a electromagnetic signal.

38. The method of claim 37, wherein each signal varies in response to the extent of the release of Ca⁺⁺.

39. The method of claim 34, wherein at least one of the first cell and the second cell includes a Ca⁺⁺ indicator.

40. The method of claim 39, wherein the Ca⁺⁺ indicator is a component effective to have a detectably altered state in the presence of Ca⁺⁺ relative to a base state in the absence of Ca⁺⁺.

41. The method of claim 40 wherein the response of said cell to changes in intracellular Ca⁺⁺ flux is measured quantitatively as a function of test substance concentration.

42. The method of claim 40, wherein the Ca⁺⁺ indicator is permeable to the membrane of at least one of the first cell and the second cell.

43. The method of claim 38, wherein the Ca⁺⁺ indicator comprises a fluorescent compound.

44. The method of claim 37, wherein each of the first and second cells includes a Ca⁺⁺ indicator.

45. The method of claim 28, wherein the first and second cells are clones.

46. The method of claim 28, wherein the test substance is selected from the group consisting of ryanodine receptor agonists, ryanodine receptor antagonists, and ryanodine receptor inverse agonists.

47. The method of claim 28, wherein the test substance binds to the second ryanodine receptor isoform.

48. The method of claim 28, wherein the ryanodine receptor activating component is selected from the group consisting of caffeine; inorganic phosphate; adenine nucleotides; adenosine; cADPR; paslitoyl carnitate; protein kinase A; calmodulin; ryanodine; methylxanthines other than caffeine; anthriquinones; digoxin; milrinone; suramin; halothine; enflurine; isoflurine; 4-chloro-m-cresol, δ-hexachlorocyclohexane; FK-506; rapamycin; bastadin 5; quinolidomicin A1; heparin; imperitoxin-a; miotoxin a; ryanotoxin; thimerisol; dithiodipyridine; hydrogen peroxide; TMPyP; disulfonic stilbene; and diethylpyrocarbonate, and derivatives and analogs of these compounds.

49. The method of claim 46, wherein the ryanodine receptor activating component is selected from the group consisting of caffeine, caffeine analogs, caffeine derivatives and mixtures thereof.

50. The method of claim 28, wherein the monitoring of at least one of steps (A) and (B) comprises detecting Ca⁺⁺ released using a CCD camera or a PMT.

51. The method of claim 28, which further comprises, after step (B), repeating step (B) in the substantial absence of the test substance.

52. The method of claim 28, which further comprises, prior to step (A), monitoring the amount of intracellular Ca⁺⁺ in the first cell in the substantial absence of the first ryanodine receptor activating component.


SEQUENCE LISTING (3 sequences)

SEQ ID NO: 1 - protein, 5038 aa, Homo sapiens
MGDAEGEDEVQFLRTDDEVVLQCSATVLKEQLKLCLAAEGFGNRLCFLEPTSNAQNVPPDLAICCFVLEQSLSVRALQEMLANTVEAGVESSQGGGHRTLLYGHAILLRHAHSRMYLSCLTTSRSMTDKLAFDVGLQEDATGEACWWTMHPASKQRSEGEKVRVGDDIILVSVSSERYLHLSTASGELQVDASFMQTLWNMNPICSRCEEGFVTGGHVLRLFHGHMDECLTISPADSDDQRRLVYYEGGAVCTHARSLWRLEPLRISWSGSHLRWGQPLRVRHVTTGQYLALTEDQGLVVVDASKAHTKATSFCFRISKEKLDVAPKRDVEGMGPPEIKYGESLCFVQHVASGLWLTYAAPDPKALRLGVLKKKAMLHQEGHMDDALSLTRCQQEESQAARMIHSTNGLYNQFIKSLDSFSGKPRGSGPPAGTALPIEGVILSLQDLIIYFEPPSEDLQHEEKQSKLRSLRNRQSLFQEEGMLSMVLNCIDRLNVYTTAAHFAEFAGEEAAESWKEIVNLLYELLASLIRGNRSNCALFSTNLDWLVSKLDRLEASSGILEVLYCVLIESPEVLNIIQENHIKSIISLLDKHGRNHKVLDVLCSLCVCNGVAVRSNQDLITENLLPGRELLLQTNLINYVTSIRPNIFVGRAEGTTQYSKWYFEVMVDEVTPFLTAQATHLRVGWALTEGYTPYPGAGEGWGGNGVGDDLYSYGFDGLHLWTGHVARPVTSPGQHLLAPEDVISCCLDLSVPSISFRINGCPVQGVFESFNLDGLFFPVVSFSAGVKVRFLLGGRHGEFKFLPPPGYAPCHEAVLPRERLHLEPIKEYRREGPRGPHLVGPSRCLSHTDFVPCPVDTVQIVLPPHLERIREKLAENIHELWALTRIEQGWTYGPVRDDNKRLHPCLVDFHSLPEPERNYNLQMSGETLKTLLALGCHVGMADEKAEDNLKKTKLPKTYMMSNGYKPAPLDLSHVRLTPAQTTLVDRLAENGHNVWARDRVGQGWSYSAVQDIPARRNPRLVPYRLLDEATKRSNRDSLCQAVRTLLGYGYNIEPPDQEPSQVENQSRCDRVRIFRAEKSYTVQSGRWYFEFEAVTTGEMRVGWARPELRPDVELGADELAYVFNGHRGQRWHLGSEPFGRPWQPGDVVGCMIDLTENTIIFTLNGEVLMSDSGSETAFREIEIGDGFLPVCSLGPGQVGHLNLGQDVSSLRFFAICGLQEGFEPFAINMQRPVTTWFSKGLPQFEPVPLEHPHYEVSRVDGTVDTPPCLRLTHRTWGSQNSLVEMLFLRLSLPVQFHQHFRCTAGATPLAPPGLQPPAEDEARAAEPDPDYENLRRSAGGWSEAENGKEGTAKEGAPGGTPQAGGEAQPARAENEKDATTEKNKKRGFLFKAKKVAMMTQPPATPTLPRLPHDVVPADNRDDPEIILNTTTYYYSVRVFAGQEPSCVWAGWVTPDYHQHDMSFDLSKVRVVTVTMGDEQGNVHSSLKCSNCYMVWGGDFVSPGQQGRISHTDLVIGCLVDLATGLMTFTANGKESNTFFQVEPNTKLFPAVFVLPTHQNVIQFELGKQKNIMPLSAAMFQSERKNPAPQCPPRLEMQMLMPVSWSRMPNHFLQVETRRAGERLGWAVQCQEPLTMMALHIPEENRCMDILELSERLDLQRFHSHTLRLYRAVCALGNNRVAHALCSHVDQAQLLHALEDAHLPGPLRAGYYDLLISIHLESACRSRRSMLSEYIVPLTPETRAITLFPPGRSTENGHPRHGLPGVGVTTSLRPPHHFSPPCFVAALPAAGAAEAPARLSPAIPLEALRDKALRMLGEAVRDGGQHARDPVGASVEFQFVPVLKLVSTLLVMGIFGDEDVKQILKMIEPEVFTEEEEEEDEEEEGEEEDEEEKEEDEEETAQEKEDEEKEEEEAAEGEKEEGLEEGLLQMKLPESVKLQMCHLLEYFCDQELQHRVESLAAFAERYVDKLQANQRSRYGLLIKAFSMTAAETARRTREFRSPPQEQINMLLQFKDGTDEEDCPLPEEIRQDLLDFHQDLLAHCGIQLDGEEEEPEEETTLGSRLMSLLEKVRLVKKKEEKPEEERSAEESKPRSLQELVSHMVVRWAQEDFVQSPELVRAMFSLLHRQYDGLGELLRALPRAYTISPSSVEDTMSLLECLGQIRSLLIVQMGPQEENLMIQSIGNIMNNKVFYQHPNLMRALGMHETVMEVMVNVLGGGESKEIRFPKMVTSCCRFLCYFCRISRQNQRSMFDHLSYLLENSGIGLGMQGSTPLDVAAASVIDNNELALALQEQDLEKVVSYLAGCGLQSCPMLVAKGYPDIGWNPCGGERYLDFLRFAVFVNGESVEENANVVVRLLIRKPECFGPALRGEGGSGLLAAIEEAIRISEDPARDGPGIRRDRRREHFGEEPPEENRVHLGHAIMSFYAALIDLLGRCAPEMHLIQAGKGEALRIRAILRSLVPLEDLVGIISLPLQIPTLGKDGALVQPKMSASFVPDHKASMVLFLDRVYGIENQDFLLHVLDVGFLPDMRAAASLDTATFSTTEMALAVNRYLCLAVLPLITKCAPLFAGTEHRAIMVDSMLHTVYRLSRGRSLTKAQRDVIEDCLMSLCRYIRPSMLQHLLRRLVFDVPILNEFAKMPLKLLTNHYERCWKYYCLPTGWANFGVTSEEELHLTRKLFWGIFDSLAHKKYDPELYRMAMPCLCAIAGALPPDYVDASYSSKAEKKATVDAEGNFDPRPVETLNVIIPEKLDSFINKFAEYTHEKWAFDKIQNNWSYGENIDEELKTHPMLRPYKTFSEKDKEIYRWPIKESLKAMIAWEWTIEKAREGEEEKTEKKKTRKISQSAQTYDPREGYNPQPPDLSAVTLSRELQAMAEQLAENYHNTWGRKKKQELEAKGGGTHPLLVPYDTLTAKEKARDREKAQELLKFLQMNGYAVTRGLKDMELDSSSIEKRFAFGFLQQLLRWMDISQEFIAHLEAVVSSGRVEKSPHEQEIKFFAKILLPLINQYFTNHCLYFLSTPAKVLGSGGHASNKEKEMITSLFCKLAALVRHRVSLFGTDAPAVVNCLHILARSLDARTVMKSGPEIVKAGLRSFFESASEDIEKMVENLRLGKVSQARTQVKGVGQNLTYTTVALLPVLTTLFQHIAQHQFGDDVILDDVQVSCYRTLCSIYSLGTTKNTYVEKLRPALGECLARLAAAMPVAFLEPQLNEYNACSVYTTKSPRERAILGLPNSVEEMCPDIPVLERLMADIGGLAESGARYTEMPHVIEITLPMLCSYLPRWWERGPEAPPSALPAGAPPPCTAVTSDHLNSLLGNILRIIVNNLGIDEASWMKRLAVFAQPIVSRARPELLQSHFIPTIGRLRKRAGKVVSEEEQLRLEAKAEAQEGELLVRDEFSVLCRDLYALYPLLIRYVDNNRAQWLTEPNPSAEELFRMVGEIFIYWSKSHNFKREEQNFVVQNEINNMSFLTADNKSKMAKAGDIQSGGSDQERTKKKRRGDRYSVQTSLIVATLKKMLPIGLNMCAPTDQDLITLAKTRYALKDTDEEVREFLHNNLHLQGKVEGSPSLRWQMALYRGVPGREEDADDPEKIVRRVQEVSAVLYYLDQTEHPYKSKKAVWHKLLSKQRRRAVVACFRMTPLYNLPTHRACNMFLESYKAAWILTEDHSFEDRMIDDLSKAGEQEEEEEEVEEKKPDPLHQLVLHFSRTALTEKSKLDEDYLYMAYADIMAKSCHLEEGGENGEAEEEVEVSFEEKQMEKQRLLYQQARLHTRGAAEMVLQMISACKGETGAMVSSTLKLGISILNGGNAEVQQKMLDYLKDKKEVGFFQSIQALMQTCSVLDLNAFERQNKAEGLGMVNEDGTVINRQNGEKVMADDEFTQDLFRFLQLLCEGHNNDFQNYLRTQTGNTTTINIIICTVDYLLRLQESISDFYWYYSGKDVIEEQGKRNFSKAMSVAKQVFNSLTEYIQGPCTGNQQSLAHSRLWDAVVGFLHVFAHMMMKLAQDSSQIELLKELLDLQKDMVVMLLSLLEGNVVNGMIARQMVDMLVESSSNVEMILKFFDMFLKLKDIVGSEAFQDYVTDPRGLISKKDFQKAMDSQKQFSGPEIQFLLSCSEADENEMINCEEFANRFQEPARDIGFNVAVLLTNLSEHVPHDPRLHNFLELAESILEYFRPYLGRIEIMGASRRIERIYFEISETNRAQWEMPQVKESKRQFIFDVVNEGGEAEKMELFVSFCEDTIFEMQIAAQISEPEGEPETDEDEGAGAAEAGAEGAEEGAAGLEGTAATAAAGATARVVAAAGRALRGLSYRSLRRRVRRLRRLTAREAATAVAALLWAAVTRAGAAGAGAAAGALGLLWGSLFGGGLVEGAKKVTVTELLAGMPDPTSDEVHGEQPAGPGGDADGEGASEGAGDAAEGAGDEEEAVHEAGPGGADGAVAVTDGGPFRPEGAGGLGDMGDTTPAEPPTPEGSPILKRKLGVDGVEEELPPEPEPEPEPELEPEKADAENGEKEEVPEPTPEPPKKQAPPSPPPKKEEAGGEFWGELEVQRVKFLNYLSRNFYTLRFLALFLAFAINFILLFYKVSDSPPGEDDMEGSAAGDVSGAGSGGSSGWGLGAGEEAEGDEDENMVYYFLEESTGYMEPALRCLSLLHTLVAFLCIIGYNCLKVPLVIFKREKELARKLEFDGLYITEQPEDDDVKGQWDRLVLNTPSFPSNYWDKFVKRKVLDKHGDIYGRERIAELLGMDLATLEITAHNERKPNPPPGLLTWLMSIDVKYQIWKFGVIFTDNSFLYLGWYMVMSLLGHYNNFFFAAHLLDIAMGVKTLRTILSSVTHNGKQLVMTVGLLAVVVYLYTVVAFNFFRKFYNKSEDEDEPDMKCDDMMTCYLFHMYVGVRAGGGIGDEIEDPAGDEYELYRVVFDITFFFFVIVILLAIIQGLIIDAFGELRDQQEQVKEDMETKCFICGIGSDYFDTTPHGFETHTLEEHNLANYMFFLMYLINKDETEHTGQESYVWKMYQERCWDFFPAGDCFRKQYEDQLS

SEQ ID NO: 2 - protein, 4967 aa, Homo sapiens
MADGGEGEDEIQFLRTDDEVVLQCTATIHKEQQKLCLAAEGFGNRLCFLESTSNSKNVPPDLSICTFVLEQSLSVRALQEMLANTVEKSEGQVDVEKWKFMMKTAQGGGHRTLLYGHAILLRHSYSGMYLCCLSTSRSSTDKLAFDVGLQEDTTGEACWWTIHPASKQRSEGEKVRVGDDLILVSVSSERYLHLSYGNGSLHVDAAFQQTLWSVAPISSGSEAAQGYLIGGDVLRLLHGHMDECLTVPSGEHGEEQRRTVHYEGGAVSVHARSLWRLETLRVAWSGSHIRWGQPFRLRHVTTGKYLSLMEDKNLLLMDKEKADVKSTAFTFRSSKEKLDVGVRKEVDGMGTSEIKYGDSVCYIQHVDTGLWLTYQSVDVKSVRMGSIQRKAIMHHEGHMDDGISLSRSQHEESRTARVIRSTVFLFNRFIRGLDALSKKAKASTVDLPIESVSLSLQDLIGYFHPPDEHLEHEDKQNRLRALKNRQNLFQEEGMINLVLECIDRLHVYSSAAHFADVAGREAGESWKSILNSLYELLAALIRGNRKNCAQFSGSLDWLISRLERLEASSGILEVLHCVLVESPEALNIIKEGHIKSIISLLDKHGRNHKVLDVLCSLCVCHGVAVRSNQHLICDNLLPGRDLLLQTRLVNHVSSMRPNIFLGVSEGSAQYKKWYYELMVDHTEPFVTAEATHLRVGWASTEGYSPYPGGGEEWGGNGVGDDLFSYGFDGLHLWSGCIARTVSSPNQHLLRTDDVISCCLDLSAPSISFRINGQPVQGMFENFNIDGLFFPVVSFSAGIKVRFLLGGRHGEFKFLPPPGYAPCYEAVLPKEKLKVEHSREYKQERTYTRDLLGPTVSLTQAAFTPIPVDTSQIVLPPHLERIREKLAENIHELWVMNKIELGWQYGPVRDDNKRQHPCLVEFSKLPEQERNYNLQMSLETLKTLLALGCHVGISDEHAEDKVKKMKLPKNYQLTSGYKPAPMDLSFIKLTPSQEAMVDKLAENAHNVWARDRIRQGWTYGIQQDVKNRRNPRLVPYTPLDDRTKKSNKDSLREAVRTLLGYGYNLEAPDQDHAARAEVCSGTGERFRIFRAEKTYAVKAGRWYFEFETVTAGDMRVGWSRPGCQPDQELGSDERAFAFDGFKAQRWHQGNEHYGRSWQAGDVVGCMVDMNEHTMMFTLNGEILLDDSGSELAFKDFDVGDGFIPVCSLGVAQVGRMNFGKDVSTLKYFTICGLQEGYEPFAVNTNRDITMWLSKRLPQFLQVPSNHEHIEVTRIDGTIDSSPCLKVTQKSFGSQNSNTDIMFYRLSMPIECAEVFSKTVAGGLPGAGLFGPKNDLEDYDADSDFEVLMKTAHGHLVPDRVDKDKEATKPEFNNHKDYAQEKPSRLKQRFLLRRTKPDYSTSHSARLTEDVLADDRDDYDFLMQTSTYYYSVRIFPGQEPANVWVGWITSDFHQYDTGFDLDRVRTVTVTLGDEKGKVHESIKRSNCYMVCAGESMSPGQGRNNNGLEIGCVVDAASGLLTFIANGKELSTYYQVEPSTKLFPAVFAQATSPNVFQFELGRIKNVMPLSAGLFKSEHKNPVPQCPPRLHVQFLSHVLWSRMPNQFLKVDVSRISERQGWLVQCLDPLQFMSLHIPEENRSVDILELTEQEELLKFHYHTLRLYSAVCALGNHRVAHALCSHVDEPQLLYAIENKYMPGLLRAGYYDLLIDIHLSSYATARLMMNNEYIVPMTEETKSITLFPDENKKHGLPGIGLSTSLRPRMQFSSPSFVSISNECYQYSPEFPLDILKSKTIQMLTEAVKEGSLHARDPVGGTTEFLFVPLIKLFYTLLIMGIFHNEDLKHILQLIEPSVFKEAATPEEESDTLEKELSVDDAKLQGAGEEEAKGGKRPKEGLLQMKLPEPVKLQMCLLLQYLCDCQVRHRIEAIVAFSDDFVAKLQDNQRFRYNEVMQALNMSAALTARKTKEFRSPPQEQINMLLNFKDDKSECPCPEEIRDQLLDFHEDLMTHCGIELDEDGSLDGNSDLTIRGRLLSLVEKVTYLKKKQAEKPVESDSKKSSTLQQLISETMVRWAQESVIEDPELVRAMFVLLHRQYDGIGGLVRALPKTYTINGVSVEDTINLLASLGQIRSLLSVRMGKEEEKLMIRGLGDIMNNKVFYQHPNLMRALGMHETVMEVMVNVLGGGESKEITFPKMVANCCRFLCYFCRISRQNQKAMFDHLSYLLENSSVGLASPAMRGSTPLDVAAASVMDNNELALALREPDLEKVVRYLAGCGLQSCQMLVSKGYPDIGWNPVEGERYLDFLRFAVFCNGESVEENANVVVRLLIRRPECFGPALRGEGGNGLLAAMEEAIKIAEDPSRDGPSPNSGSSKTLDTEEEEDDTIHMGNAIMTFYSALIDLLGRCAPEMHLIHAGKGEAIRIRSILRSLIPLGDLVGVISIAFQMPTIAKDGNVVEPDMSAGFCPDHKAAMVLFLDRVYGIEVQDFLLHLLEVGFLPDLRAAASLDTAALSATDMALALNRYLCTAVLPLLTRCAPLFAGTEHHASLIDSLLHTVYRLSKGCSLTKAQRDSIEVCLLSICGQLRPSMMQHLLRRLVFDVPLLNEHAKMPLKLLTNHYERCWKYYCLPGGWGNFGAASEEELHLSRKLFWGIFDALSQKKYEQELFKLALPCLSAVAGALPPDYMESNYVSMMEKQSSMDSEGNFNPQPVDTSNITIPEKLEYFINKYAEHSHDKWSMDKLANGWIYGEIYSDSSKVQPLMKPYKLLSEKEKEIYRWPIKESLKTMLARTMRTERTREGDSMALYNRTRRISQTSQVSVDAAHGYSPRAIDMSNVTLSRDLHAMAEMMAENYHNIWAKKKKMELESKGGGNHPLLVPYDTLTAKEKAKDREKAQDILKFLQINGYAVSRGFKDLELDTPSIEKRFAYSFLQQLIRYVDEAHQYILEFDGGSRGKGEHFPYEQEIKFFAKVVLPLIDQYFKNHRLYFLSAASRPLCSGGHASNKEKEMVTSLFCKLGVLVRHRISLFGNDATSIVNCLHILGQTLDARTVMKTGLESVKSALRAFLDNAAEDLEKTMENLKQGQFTHTRNQPKGVTQIINYTTVALLPMLSSLFEHIGQHQFGEDLILEDVQVSCYRILTSLYALGTSKSIYVERQRSALGECLAAFAGAFPVAFLETHLDKHNIYSIYNTKSSRERAALSLPTNVEDVCPNIPSLEKLMEEIVELAESGIRYTQMPHVMEVILPMLCSYMSRWWEHGPENNPERAEMCCTALNSEHMNTLLGNILKIIYNNLGIDEGAWMKRLAVFSQPIINKVKPQLLKTHFLPLMEKLKKKAATVVSEEDHLKAEARGDMSEAELLILDEFTTLARDLYAFYPLLIRFVDYNRAKWLKEPNPEAEELFRMVAEVFIYWSKSHNFKREEQNFVVQNEINNMSFLITDTKSKMSKAAVSDQERKKMKRKGDRYSMQTSLIVAALKRLLPIGLNICAPGDQELIALAKNRFSLKDTEDEVRDIIRSNIHLQGKLEDPAIRWQMALYKDLPNRTDDTSDPEKTVERVLDIANVLFHLEQKSKRVGRRHYCLVEHPQRSKKAVWHKLLSKQRKRAVVACFRMAPLYNLPRHRAVNLFLQGYEKSWIETEEHYFEDKLIEDLAKPGAEPPEEDEGTKRVDPLHQLILLFSRTALTEKCKLEEDFLYMAYADIMAKSCHDEEDDDGEEEVKSFEEKEMEKQKLLYQQARLHDRGAAEMVLQTISASKGETGPMVAATLKLGIAILNGGNSTVQQKMLDYLKEKKDVGFFQSLAGLMQSCSVLDLNAFERQNKAEGLGMVTEEGSGEKVLQDDEFTCDLFRFLQLLCEGHNSDFQNYLRTQTGNNTTVNIIISTVDYLLRVQESISDFYWYYSGKDVIDEQGQRNFSKAIQVAKQVFNTLTEYIQGPCTGNQQSLAHSRLWDAVVGFLHVFAHMQMKLSQDSSQIELLKELMDLQKDMVVMLLSMLEGNVVNGTIGKQMVDMLVESSNNVEMILKFFDMFLKLKDLTSSDTFKEYDPDGKGVISKRDFHKAMESHKHYTQSETEFLLSCAETDENETLDYEEFVKRFHEPAKDIGFNVAVLLTNLSEHMPNDTRLQTFLELAESVLNYFQPFLGRIEIMGSAKRIERVYFEISESSRTQWEKPQVKESKRQFIFDVVNEGGEKEKMELFVNFCEDTIFEMQLAAQISESDLNERSANKEESEKERPEEQGPRMAFFSILTVRSALFALRYNILTLMRMLSLKSLKKQMKKVKKMTVKDMVTAFFSSYWSIFMTLLHFVASVFRGFFRIICSLLLGGSLVEGAKKIKVAELLANMPDPTQDEVRGDGEEGERKPLEAALPSEDLTDLKELTEESDLLSDIFGLDLKREGGQYKLIPHNPNAGLSDLMSNPVPMPEVQEKFQEQKAKEEEKEEKEETKSEPEKAEGEDGEKEEKAKEDKGKQKLRQLHTHRYGEPEVPESAFWKKIIAYQQKLLNYFARNFYNMRMLALFVAFAINFILLFYKVSTSSVVEGKELPTRSSSENAKVTSLDSSSHRIIAVHYVLEESSGYMEPTLRILAILHTVISFFCIIGYYCLKVPLVIFKREKEVARKLEFDGLYITEQPSEDDIKGQWDRLVINTQSFPNNYWDKFVKRKVMDKYGEFYGRDRISELLGMDKAALDFSDAREKKKPKKDSSLSAVLNSIDVKYQMWKLGVVFTDNSFLYLAWYMTMSVLGHYNNFFFAAHLLDIAMGFKTLRTILSSVTHNGKQLVLTVGLLAVVVYLYTVVAFNFFRKFYNKSEDGDTPDMKCDDMLTCYMFHMYVGVRAGGGIGDEIEDPAGDEYEIYRIIFDITFFFFVIVILLAIIQGLIIDAFGELRDQQEQVKEDMETKCFICGIGNDYFDTVPHGFETHTLQEHNLANYLFFLMYLINKDETEHTGQESYVWKMYQERCWEFFPAGDCFRKQYEDQLN

SEQ ID NO: 3 - protein, 4870 aa, Homo sapiens
MAEGGEGGEDEIQFLRTEDEVVLQCIATIHKEQRKFCLAAEGLGNRLCFLEPTSEAKYIPPDLCVCNFVLEQSLSVRALQEMLANTGENGGEGAAQGGGHRTLLYGHAVLLRHSFSGMYLTCLTTSRSQTDKLAFDVGLREHATGEACWWTIHPASKQRSEGEKVRIGDDLILVSVSSERYLHLSVSNGNIQVDASFMQTLWNVHPTCSGSSIEEGYLLGGHVVRLFHGHDECLTIPSTDQNDSQHRRIFYEAGGAGTRARSLWRVEPLRISWSGSNIRWGQAFRLRHLTTGHYLALTEDQGLILQDRAKSDTKSTAFSFRASKELKEKLDSSHKRDIEGMGVPEIKYGDSVCFVQHIASGLWVTYKAQDAKTSRLGPLKRKVILHQEGHMDDGLTLQRCQREESQAARIIRNTTALFSQFVSGNNRTAAPITLPIEEVLQTLQDLIAYFQPPEEEMRHEDKQNKLRSLKNRQNLFKEEGMLALVLNCIDRLNVYNSVAHFAGIAREESGMAWKEILNLLYKLLAALIRGNRNNCAQFSNNLDWLISKLDRLESSSGILEVLHCILTESPEALNLIAEGHIKSIISLLDKHGRNHKVLDILCSLCLCNGVAVRANQNLICDNLLPRRNLLLQTRLINDVTSIRPNIFLGVAEGSAQYKKWYFELIIDQVDPFLTAEPTHLRVGWASSSGYAPYPGGGEGWGGNGVGDDLYSYGFDGLHLWSGRIPRAVASINQHLLRSDDVVSCCLDLGVPSISFRINGQPVQGMFENFNTDGLFFPVMSFSAGVKVRFLMGGRHGEFKFLPPSGYAPCYEALLPKEKMRLEPVKEYKRDADGIRDLLGTTQFLSQASFIPCPVDTSQVILPPHLEKIRDRLAENIHELWGMNKIELGWTFGKIRDDNKRQHPCLVEFSKLPETEKNYNLQMSTETLKTLLALGCHIAHVNPAAEEDLKKVKLPKNYMMSNGYKPAPLDLSDVKLLPPQEILVDKLAENAHNVWAKDRIKQGWTYGIQQDLKNKRNPRLVPYALLDERTKKSNRDSLREAVRTFVGYGYNIEPSDQELADSAVEKVSIDKIRFFRVERSYAVRSGKWYFEFEVVTGGDMRVGWARPGCRPDVELGADDQAFVFEGNRGQRWHQGSGYFGRTWQPGDVVGCMINLDDASMIFTLNGELLITNKGSELAFADYEIENGFVPICCLGLSQIGRMNLGTDASTFKFYTMCGLQEGFEPFAVNMNRDVAMWFSKRLPTFVNVPKDHPHIEVMRIDGTMDSPPCLKVTHKTFGTQNSNADMIYCRLSMPVECHSSFSHSPCLDSEAFQKRKQMQEILSHTTTQCYYAIRIFAGQDPSCVWVGWVTPDYHLYSEKFDLNKNCTVTVTLGDERGRVHESVKRSNCYMVWGGDIVASSQRSNRSNVDLEIGCLVDLAMGMLSFSANGKELGTCYQVEPNTKVFPAVFLQPTSTSLFQFELGKLKNAMPLSAAIFRSEEKNPVPQCPPRLDVQTIQPVLWSRMPNSFLKVETERVSERHGWVVQCLEPLQMMALHIPEENRCVDILELCEQEDLMRFHYHTLRLYSAVCALGNSRVAYALCSHVDLSQLFYAIDNKYLPGLLRSGFYDLLISIHLASAKERKLMMKNEYIIPITSTTRNICLFPDESKRHGLPGVGLRTCLKPGFRFSTPCFVVTGEDHQKQSPEIPLESLRTKALSMLTEAVQCSGAHIRDPVGGSVEFQFVPVLKLIGTLLVMGVFDDDDVRQILLLIDPSVFGEHSAGTEEGAEKEEVTQVEEKAVEAGEKAGKEAPVKGLLQTRLPESVKLQMCELLSYLCDCELQHRVEAIVAFGDIYVSKLQANQKFRYNELMQALNMSAALTARKTKEFRSPPQEQINMLLNFQLGENCPCPEEIREELYDFHEDLLLHCGVPLEEEEEEEEDTSWTGKLCALVYKIKGPPKPEKEQPTEEEERCPTTLKELISQTMICWAQEDQIQDSELVRMMFNLLRRQYDSIGELLQALRKTYTISHTSVSDTINLLAALGQIRSLLSVRMGKEEELLMINGLGDIMNNKVFYQHPNLMRVLGMHETVMEVMVNVLGTEKSQIAFPKMVASCCRFLCYFCRISRQNQKAMFEHLSYLLENSSVGLASPSMRGSTPLDVAASSVMDNNELALSLEEPDLEKVVTYLAGCGLQSCPMLLAKGYPDVGWNPIEGERYLSFLRFAVFVNSESVEENASVVVKLLIRRPECFGPALRGEGGNGLLAAMQGAIKISENPALDLPSQGYKREVSTEDDEEEEEIVHMGNAIMSFYSALIDLLGRCAPEMHLIQTGKGEAIRIRSILRSLVPTEDLVGIISIPLKLPSLNKDGSVSEPDMAANFCPDHKAPMVLFLDRVYGIKDQTFLLHLLEVGFLPDLRASASLDTVSLSTTEAALALNRYICSAVLPLLTRCAPLFAGTEHCTSLIDSTLQTIYRLSKGRSLTKAQRDTIEECLLAICNHLRPSMLQQLLRRLVFDVPQLNEYCKMPLKLLTNHYEQCWKYYCLPSGWGSYGLAVEEELHLTEKLFWGIFDSLSHKKYDPDLFRMALPCLSAIAGALPPDYLDTRITATLEKQISVDADGNFDPKPINTMNFSLPEKLEYIVTKYAEHSHDKWACDKSQSGWKYGISLDENVKTHPLIRPFKTLTEKEKEIYRWPARESLKTMLAVGWTVERTKEGEALVQQRENEKLRSVSQANQGNSYSPAPLDLSNVVLSRELQGMVEVVAENYHNIWAKKKKLELESKGGGSHPLLVPYDTLTAKEKFKDREKAQDLFKFLQVNGIIVSRGMKDMELDASSMEKRFAYKFLKKILKYVDSAQEFIAHLEAIVSSGKTEKSPRDQEIKFFAKVLLPLVDQYFTSHCLYFLSSPLKPLSSSGYASHKEKEMVAGLFCKLAALVRHRISLFGSDSTTMVSCLHILAQTLDTRTVMKSGSELVKAGLRAFFENAAEDLEKTSENLKLGKFTHSRTQIKGVSQNINYTTVALLPILTSIFEHVTQHQFGMDLLLGDVQISCYHILCSLYSLGTGKNIYVERQRPALGECLASLAAAIPVAFLEPTLNRYNPLSVFNTKTPRERSILGMPDTVEDMCPDIPQLEGLMKEINDLAESGARYTEMPHVIEVILPMLCNYLSYWWERGPENLPPSTGPCCTKVTSEHLSLILGNILKIINNNLGIDEASWMKRIAVYAQPIISKARPDLLRSHFIPTLEKLKKKAVKTVQEEEQLKADGKGDTQEAELLILDEFAVLCRDLYAFYPMLIRYVDNNRSNWLKSPDADSDQLFRMVAEVFILWCKSHNFKREEQNFVIQNEINNLAFLTGDSKSKMSKAMQVKSGGQDQERKKTKRRGDLYSIQTSLIVAALKKMLPIGLNMCTPGDQELISLAKSRYSHRDTDEEVREHLRNNLHLQEKSDDPAVKWQLNLYKDVLKSEEPFNPEKTVERVQRISAAVFHLEQVEQPLRSKKAVWHKLLSKQRKRAVVACFRMAPLYNLPRHRSINLFLHGYQRFWIETEEYSFEEKLVQDLAKSPKVEEEEEEETEKQPDPLHQIILYFSRNALTERSKLEDDPLYTSYSSMMAKSCQSGEDEEEDEDKEKTFEEKEMEKQKTLYQQARLHERGAAEMVLQMISASKGEMSPMVVETLKLGIAILNGGNAGVQQKMLDYLKEKKDAGFFQSLSGLMQSCSVLDLNAFERQNKAEGLGMVTEEGTLIVRERGEKVLQNDEFTRDLFRFLQLLCEGHNSDFQNFLRTQMGNTTTVNVIISTVDYLLRLQESISDFYWYYSGKDIIDESGQHNFSKALAVTKQIFNSLTEYIQGPCIGNQQSLAHSRLWDAVVGFLHVFANMQMKLSQDSSQIELLKELLDLLQDMVVMLLSLLEGNVVNGTIGKQMVDTLVESSTNVEMILKFFDMFLKLKDLTSSDTFKEYDPDGKGIISKKEFQKAMEGQKQYTQSEIDFLLSCAEADENDMFNYVDFVDRFHEPAKDIGFNVAVLLTNLSEHMPNDSRLKCLLDPAESVLNYFEPYLGRIEIMGGAKKIERVYFEISESSRTQWEKPQVKESKRQFIFDVVNEGGEQEKMELFVNFCEDTIFEMQLASQISESDSADRPEEEEEDEDSSYVLEIAGEEEEDGSLEPASAFAMACASVKRNVTDFLKRATLKNLRKQYRNVKKMTAKELVKVLFSFFWMLFVGLFQLLFTILGGIFQILWSTVFGGGLVEGAKNIRVTKILGDMPDPTQFGIHDDTMEAERAEVMEPGITTELVHFIKGEKGDTDIMSDLFGLHPKKEGSLKHGPEVGLGDLSEIIGKDEPPTLESTVQKKRKAQAAEMKAANEAEGKVESEKADMEDGEKEDKDKEEEQAEYLWTEVTKKKKRRCGQKVEKPEAFTANFFKGLEIYQTKLLHYLARNFYNLRFLALFVAFAINFILLFYKVTEEPLEEETEDVANLWNSFNDEEEEEAMVFFVLQESTGYMAPTLRALAIIHTIISLVCVVGYYCLKVPLVVFKREKEIARKLEFDGLYITEQPSEDDIKGQWDRLVINTPSFPNNYWDKFVKRKVINKYGDLYGAERIAELLGLDKNALDFSPVEETKAEAASLVSWLSSIDMKYHIWKLGVVFTDNSFLYLAWYTTMSVLGHYNNFFFAAHLLDIAMGFKTLRTILSSVTHNGKQLVLTVGLLAVVVYLYTVVAFNFFRKFYNKSEDDDEPDMKCDDMMTCYLFHMYVGVRAGGGIGDEIEDPAGDPYEMYRIVFDITFFFFVIVILLAIIQGLIIDAFGELRDQQEQVREDMETKCFICGIGNDYFDTTPHGFETHTLQEHNLANYLFFLMYLINKDETEHTGQESYVWKMYQERCWDFFPAGDCFRKQYEDQLG